(19) United States
(12) Patent Application Publication
(10) Pub. No.: US 2024/0245859 A1
(43) Pub. Date: Jul. 25, 2024

(54) AUTOMATIC GRAVITY INFUSION SYSTEM

(71) Applicant: Think N Kreate Limited, Hong Kong (HK)

(72) Inventor: Choi Ting LAU, Hong Kong (HK)

(73) Assignee: Think N Kreate Limited, Hong Kong (HK)

(21) Appl. No.: 18/406,558

(22) Filed: Jan. 8, 2024

Related U.S. Application Data
(60) Provisional application No. 63/480,599, filed on Jan. 19, 2023.

(51) Int. Cl. A61M 5/172; A61M 5/142; A61M 5/168

(52) U.S. Cl. CPC A61M 5/1723; A61M 5/14232; A61M 5/16813; A61M 2202/0486

(57) ABSTRACT

An automated, closed-loop gravity infusion system including a fluid source, a drop counter operatively engaged with a drip chamber of the fluid source, a roller clamp functionally linked with the drop counter, a processor operably engaged with the roller clamp, and a human-machine interface (HMI) functionally linked with the processor. Patient data is accessed by the HMI. The system utilizes the patient data and data from the drop counter to determine an appropriate roller clamp position. The drop counter continuously monitors the drip chamber's drip rate and feeds the data to the processor which then analyzes the data and automatically adjusts the roller clamp's position to provide an appropriate fluid dosage to the patient. The HMI is functionally linked to a remote centralized computing system configured to simultaneously monitor several identical systems, each of which is being used to infuse a different patient.

### REFERENCE TO RELATED APPLICATIONS

This application claims the benefit of U.S. Provisional Patent Application Ser. No. 63/480,599 filed Jan. 19, 2023, the entire disclosure of which is incorporated herein by reference.

### TECHNICAL FIELD

This disclosure is directed to medical equipment. In particular the disclosure relates to medical equipment used to intravenously deliver fluids to a patient. Specifically, the disclosure relates to a system comprising a fluid source, a drop counter operatively engaged with a drip chamber of the fluid source, a roller clamp functionally linked with the drop counter, a processor operably engaged with the roller clamp, and a human-machine interface (HMI) functionally linked with the processor. Patient data is accessed by the HMI and the processor utilizes the patient data and real time drop counter data to determine and automatically set an appropriate roller clamp position in order to automatically deliver a prescribed fluid dosage to the patient under force of gravity. The system is configured to mimic typical human interaction with a traditional roller clamp as closely as possible.

### BACKGROUND ART

Intravenous (IV) infusion therapy is a widely used technique for delivering fluids, nutrients, vitamins and other substances directly into a patient's bloodstream. The fluid to be administered to the patient is contained in an intravenous bag (IV) which is suspended from a pole. A drip chamber extends outwardly from the IV bag and tubing extends from the drip chamber to a luer (i.e., hollow tube/needle) which is inserted directly into a patient's vein. During use, fluid drips from the bag into the drip chamber under force of gravity. The liquid then flows down the tube to the luer under force of gravity and ultimately flows into the patient's bloodstream.

It is needful to control the rate of flow of the fluid from the IV bag to the patient. There are two main types of device used for this purpose, infusion pumps and “gravity infusion devices” or “gravity IVs”. Infusion pumps deliver precise fluid flow rates to the patient but tend to be relatively expensive and require dedicated valve and pump disposables. In other words, the valve and pump disposables are designed for use with only a single type of infusion pump and cannot be operated with other types of valves and other pump disposables. The cost of infusion pumps and their required dedicated equipment makes it difficult for medical facilities in less affluent communities to provide such equipment for use. Gravity IV systems on the other hand do not require dedicated peripheral equipment and are less expensive to purchase and operate. The most basic flow control device used in gravity IV systems is the roller clamp. Roller clamps are engaged with the tubing between the drip chamber and the luer, and the position of the roller clamp on the tubing may be adjusted as needed. Roller clamps can be used with any type of IV bag, drip chamber, and tubing and do not require any dedicated equipment for use. Roller clamps are therefore suitable for use in any medical environment.

One of the issues with intravenous infusion is that it is needful for the delivery of fluid from the IV bag to the luer to be at a safe and steady rate, and particularly at a flow rate prescribed by a doctor. Currently-known IV pumps and gravity IVs are open-loop systems, i.e., systems which do not provide immediate feedback to practitioners on how the system is actually performing. The motion of fluid through the system is pre-determined, based on original design calculations. In other words, currently-known IV pumps do not monitor fluid flow and feedback to the control unit. They only operate based on pre-determined design values instead of real-time data. If there are no manufacturing variations, then IV pumps can be very accurate and gravity IV systems may be reasonably accurate but less precise than an IV pump. However, if there are manufacturing variants in an infusion system including an IV pump or a gravity IV, the system may not actually deliver the calculated volume of fluid to the patient. For example, an IV pump may move ten motor steps, but this does not automatically mean that the dedicated disposables used with the pump have dispensed the exact dosage of fluid. Manufacturing variations and tolerances in the dedicated disposables may negatively impact fluid flow and therefore affect fluid delivery to the patient. Practitioners may not be aware that the exact dosage of fluid has not been dispensed to the patient because there is no mechanism in currently-known infusion systems to monitor actual fluid flow to a patient's body.

Typically roller clamps include a body which has an internal passageway through which the tubing extends. A roller wheel is operatively engaged with the body, extends at least partially into the passageway, and will contact the tubing extending therethrough. The passageway may be arranged in the body to follow a pathway which causes the roller wheel to be moved closer to or further away from the tubing. Alternatively, an internal wall of the roller clamp, which defines the internal passageway, may narrow in a direction moving away from a first end of the body and towards a second end of the body. A medical practitioner will adjust the position of the roller wheel relative to the roller clamp body in order to control fluid flow through the tubing. The practitioner will simultaneously and manually push and roll the roller wheel along the passageway in one of a first direction or a second direction. As the roller wheel is moved in the first direction, the roller wheel progressively narrows the diameter of the tubing extending through the passageway, and thereby decreases the flow rate of the fluid moving through the tubing. The roller wheel may be moved in the first direction to a degree sufficient to totally stop the flow of liquid through the tubing. If the medical practitioner decides it is necessary to increase the rate of infusion, he or she moves the roller wheel in the second direction relative to the first end of the body of the roller clamp. This rotation and movement of the roller wheel in the second direction will reduce the tendency for the roller wheel to narrow or pinch the tubing. The diameter of the tubing will therefore effectively increase, and flow rate of fluid through the tubing will tend to increase.

Typically, if the roller wheel of a roller clamp is manually rolled towards the first end of the roller clamp body, i.e., in the direction moving towards the IV bag, then the flow rate through the tubing will increase. If the roller wheel is manually rolled towards the second end of the roller clamp body, i.e., in a direction moving along the tubing and towards the luer thereon, then the flow rate through the tubing will decrease.

In currently-known gravity IV systems the flow rate therethrough is manually set as described above. The practitioner will first calculate the required fluid flow rate using a patient's prescription. The practitioner will then determine the actual flow rate of the fluid from the IV bag by physically counting the drops of fluid dripping from the IV bag into the drip chamber over a period of time measured with a watch or another timing device. The observed number of drops over the preset period of time is then compared with the required or desired fluid flow rate. If the observed number of drops which are dripping into the drip chamber in a certain time is too high, the practitioner will manually adjust the roller clamp by rolling the roller wheel towards the luer at the patient-end of the tubing to effectively narrow the diameter of the tubing and thereby slow down the rate of travel of the fluid through the tubing. If the observed number of drops dripping into the drip chamber in a certain time is too low, then the practitioner will manually roll the roller wheel towards the IV bag to effectively increase the diameter of the tubing and thereby to increase the flow rate through the tubing.

As stated previously herein, one of the advantages of gravity IV systems is that they are relatively inexpensive. They are also easy to set up and are easy to operate. One of disadvantages of gravity IV systems is that they are relatively imprecise. If the practitioner is in a hurry or is slightly distracted they may not accurately count the number of drops dripping in the drip chamber and will therefore not arrive at the correct fluid flow rate for delivery to the patient. Furthermore, since the practitioner sets the position of the roller wheel by finger touch, if the roller wheel is not moved to the optimum position on the roller body, the fluid flow through the tubing will not be set at the optimum rate. Additionally, after initial setting of the roller, the position of the roller may change because of a number of factors, including but not limited to patient movement, tubing creep, and/or IV bag volume changes. The manual adjustment of fluid flow rate from currently-known gravity IVs therefore requires the practitioner's close attention and the flow rate may have to be monitored and adjusted several times to deliver the prescribed amount of fluid to the patient.

Various infusion systems are described in the prior art. AU2020250300(Peret) for example, describes a system, method and apparatus for monitoring, regulating, or controlling fluid flow. The reference discloses a closed loop system which includes a drop counter having optical sensors that monitor the rate of fluid dripping from an IV bag into a drip chamber. In one embodiment, the sensors relay data to a processor and the processor controls a motor which adjusts the position of interacting members relative to a roller clamp (referred to in the publication as a “valve”). The flow rate sensor uses images to estimate flow through the drip chamber and then controls the valve (roller clamp) based on the estimated flow rate. The reference discloses that a motor is coupled to a lead screw mechanism to control the roller clamp via interaction with the interacting members. When the motor is actuated, the interacting members are moved and therefore a roller wheel of the roller clamp is moved. The movement of the interacting members will push the roller wheel in order to move the roller wheel relative to the roller clamp's body. This pushing action will tend to damage the roller wheel and/or the body of the roller clamp as repeated adjustments are made to control the fluid flow through the tubing. Roller clamps are design for human interaction and manipulation, and not for machine operation. Operating roller clamps using machines tends to over-stress the roller wheel shaft and its housing and will lead to premature failure of the roller clamp. Failure of the roller clamp during delivery of fluids to a patient will of course, negatively impact that fluid delivery and could be harmful to the patient.

AU2020250300 also discloses that an electronic device such as a smart phone or computer being used to control the system remotely and that the flow meter may alarm when the system detects free flow conditions, determines if the flow rate is greater than a predetermined threshold, or is outside a predetermined range, and/or if any abnormal behavior is detected. Since the disclosed system uses images of the flow counter to control fluid flow rate, it is unclear how the flow counter could differentiate between abnormalities and reverse flow of the fluid through the tubing. Furthermore, while AU2020250300 discloses controlling fluid flow via variety of new disposables, these disposables interact with the tubing of an IV system in new ways. These interactions between the new disposables and the tubing increase the uncertainty of exactly how fluid flows inside the tubing and therefore require advanced engineering and testing in order to be able to accurately predict and control fluid flow with the disclosed system.

### SUMMARY OF THE INVENTION

The closed-loop gravity infusion system and method disclosed herein provides a system and method of utilizing a range of differently configured, currently known roller clamps to precisely and automatically deliver fluid at a prescribed flow rate to a patient. The loading and control mechanism of the disclosed system is capable of accommodating different roller clamp designs and is configured to mimic typical human interaction with traditional roller clamps as closely as possible. The disclosed system and method can turn the simplest IV gravity set from an inaccurate, imprecise gravity IV set to a precision gravity IV set without the need for a high-end IV pump and dedicated peripherals. Furthermore, the system disclosed herein does not introduce any new interactions between the roller clamp and the IV tubing to control fluid flow and therefore retains the well understood fluid flow through tubing relating to traditional roller clamps. Additionally, the presently-disclosed gravity infusion system monitors fluid flow in real time and provides immediate feedback to the control unit. The presently-disclosed gravity infusion system therefore provides real time feedback to practitioners.

The system of the present disclosure includes a fluid source and a drop counter operatively engaged with a drip chamber of the fluid source. The drop counter is configured to automatically count drops of fluid dripping from the fluid source into the drip chamber. The system further includes any traditional roller clamp retained within a holster provided in a housing, with the roller clamp being functionally linked with the drop counter. A processor is operably engaged with the roller clamp and a human-machine interface (HMI) is functionally linked with the processor. Patient-specific data, including the patient's prescription for fluid infusion, is entered into the HMI via a user interface or is uploaded from a database. Programming provided in the processor utilizes the patient data from the HMI and the drip data from the drop counter to calculate a fluid flow rate to the patient which will meet the criteria of the patient's prescription. The programming is also configured to determine an appropriate position for the roller wheel of the roller clamp in order to attain the desired flow rate and then to automatically adjust the position of the roller wheel relative to the tubing in order to cause the desired rate of fluid to be delivered through the tubing to the patient. The drop counter continuously monitors the drip chamber's drip rate in real time and feeds the data to the processor. The processor analyzes the fed data in real time and automatically adjusts, in real time, the position of the roller wheel in the roller clamp in order to provide an appropriate dosage of fluid to a patient. The HMI may be functionally linked to a remote centralized computing system which is configured to simultaneously monitor several identical infusion systems, each being used to infuse a different patient.

The presently disclosed automatically-controlled gravity IV provides a cost-effective solution to replace previously known gravity IVs and infusion pumps and provides a cost-effective and more accurate method for delivering IV therapy with a system which includes a traditional roller clamp. One reason the presently-disclosed infusion system offers greater accuracy than previously known gravity IVs is because the presently disclosed system is a closed-loop system. The presently-disclosed system provides immediate feedback to practitioners, whereas PRIOR ART infusion systems do not. Furthermore, the presently disclosed infusion system enables a single practitioner to monitor and control multiple infusion devices in real time from a remote, centralized computer system.

In one aspect, an exemplary embodiment of the present disclosure may provide an infusion system comprising a drop counter adapted to be engaged with a drip chamber of a fluid source, wherein the fluid source is configured to deliver a fluid to a patient's body through tubing under force of gravity; a roller clamp functionally linked to the drop counter; an adjustment assembly operably engaged with a roller of the roller clamp, said adjustment assembly including at least one spring-loaded plunger which urges the roller towards the tubing; and a processor provided with programming configured to automatically adjust a position of the roller relative to the roller clamp via the adjustment assembly and at least partially in response to drip data gathered by the drop counter.

In one embodiment, the at least one spring-loaded plunger may comprise a first plunger and a second plunger which engage spaced apart locations on the roller. In one embodiment, the first plunger and the second plunger may be independently operable. In one embodiment, each of the first plunger and the second plunger may include a pin roller which contacts a circumferential surface of the roller. In one embodiment, wherein an exterior surface of the pin roller which contacts the circumferential surface of the roller may include a resilient material. In one embodiment, the first plunger and the second plunger, together, may move the roller relative to the roller clamp. In one embodiment, the first plunger, and the second plunger, together, may push and rotate the roller relative to the roller clamp.

In one embodiment, the infusion may further comprise a strain gauge which measures forces experienced by the first plunger and the second plunger. In one embodiment, the infusion system may further comprise at least one limit switch which limits travel of the adjustment assembly relative to the roller clamp. In one embodiment, the infusion system may further comprise a counter which counts how many times a position of the roller relative to the roller clamp is adjusted by the adjustment assembly. In one embodiment, the infusion system may further comprise a housing with a holster provided therein, wherein the holster is configured to receive any one of a variety of differently configured roller clamps therein. In one embodiment, the infusion system may further comprise a jogging mechanism configured to move a deformed length of tubing out of the roller clamp and to introduce a new un-deformed length of tubing into the roller clamp.

In one embodiment, the infusion system may be a closed-loop system. In one embodiment, the fluid source may include an intravenous bag adapted to contain a volume of the fluid and the drip chamber is operatively engaged with the intravenous bag; and wherein the drop counter is configured to automatically count drops of fluid falling into the drip chamber from the intravenous bag and feed drip data to the processor in real time.

In one embodiment, the infusion system may further comprise a human-machine interface (HMI) functionally linked with the processor, said HMI being configured to enable access patient data. In one embodiment, the infusion system may further comprise a user interface provided on the HMI, and the HMI may access patient data through entry of information via the user interface. In one embodiment, the infusion system may further comprise a database having patient data stored therein, and wherein the HMI may access the patient data via automatic retrieval of the patient data from the database. In one embodiment, the infusion system may further comprise a remote computing system functionally linked with the HMI. In one embodiment, the infusion system may further comprise a camera provided on the drop counter, said camera being configured to capture one or both of drip data and fluid level within a drip chamber of the drop counter.

In another aspect, an exemplary embodiment of the present disclosure may provide an infusion system comprising an intravenous bag adapted to hold a volume of fluid to be delivered to a patient; tubing extending between the intravenous bag and the patient's body; a drip chamber engaged with the tubing at a location between the intravenous bag and the patient's body; a drop counter operably engaged with the drip chamber, said drop counter being configured to automatically determine a number of drops of fluid entering the drip chamber from the intravenous bag in real time; a roller clamp positioned between the drip chamber and the patient's body, wherein the tubing extends through a bore of the roller clamp; a roller provided in the roller clamp, wherein the roller bears upon the tubing extending through the bore of the roller clamp; a first plunger and a second plunger which contact the roller at spaced-apart locations from one another, said first plunger and the second plunger being operable to move the roller relative to the roller clamp; and a processor functionally linked to drop counter and the roller, said processor automatically controlling movement of the roller relative to the tubing to control a flow rate of fluid through the tubing in response to drip data fed by the drop counter to the processor.

In one embodiment, the first plunger and the second plunger may be independently operable. In one embodiment, the infusion system may further comprise a human-machine interface (HMI) functionally linked with the processor, said HMI being configured to access patient data and provide the same to the processor. In one embodiment, the infusion system may further comprise a remote computing system functionally linked with the HMI. In one embodiment, the infusion system may further comprise a camera provided on the drop counter, wherein the camera is configured to capture images of the number of drops of fluid entering a drip chamber of the drop counter and or a fluid level within the drip chamber.

In another aspect, an exemplary embodiment of the present disclosure may provide a method of controlling a flow rate of a fluid to a patient using an infusion system which includes tubing extending from an intravenous bag; said method comprising accessing patient data via a human-machine interface (HMI) of the infusion system; receiving, at a processor of the infusion system, drip rate data from a drop counter of the infusion system; analyzing the patient data and the drip rate data with the processor; determining, with the processor, a desired flow rate of fluid based on a desired dosage of fluid to be delivered by the infusion system to the patient over a period of time based on the analysis of the patient data and drip rate data; automatically adjusting a position of a roller of a roller clamp of the infusion system using a drive mechanism functionally linked to the processor to push and rotate the roller relative to the roller clamp; and delivering the desired dosage of fluid to the patient through the tubing.

In one embodiment, automatically adjusting a position of a roller clamp may include rotating a roller of the roller clamp in one of a first direction and a second direction; contacting the tubing which extends through a bore of the roller clamp with the roller; changing a size of the bore of the tubing as the roller rotates in the one of the first direction and the second direction; and changing the flow rate of the fluid through the tubing to the desired flow rate as the size of the bore is changed. In one embodiment, wherein receiving drip rate data from the drop counter may include automatically counting, with a sensor of the drop counter, a number of drops of fluid entering a drip chamber from an intravenous bag in real time; communicating the counted number of drops of fluid from the drop counter to the processor; determining, with programming in the processor, a flow rate of fluid moving through the tubing from the drip chamber; and automatically adjusting the position of the roller relative to the clamp without human intervention and in real time.

In one embodiment, wherein receiving drip rate data from the drop counter may include capturing images of drops of fluid entering the drip chamber of the drop counter with a camera; and determining the drip rate data from the captured images. In one embodiment, the method may further comprise capturing images of an actual level of fluid within a drip chamber of the drop counter; and determining when the actual level of fluid rises above a threshold fluid level. In one embodiment, the method may further comprise determining backflow within the drip chamber when the actual level of fluid rises above the threshold fluid level; and issuing an alarm. In one embodiment, issuing the alarm may include one or more of issuing an audible alert, issuing a visual alert, and feeding a real-time image of the actual level of fluid in the drip chamber to a display screen of the HMI. In one embodiment, the method may further comprise providing a sensor for detecting one of backflow, bubbles in the fluid, air in the fluid, and changes in pressure in the tubing; and generating one or both of an audible alarm and a visual alarm when the sensor detects the one of backflow, bubbles in the fluid, air in the fluid, high pressure in the tubing and a low pressure in the tubing. In one embodiment, accessing patient data via the HMI may include automatically retrieving patient data stored in a database. In one embodiment, accessing patient data via the HMI may include entering patient data into the HMI via a user interface.

In another aspect, and exemplary embodiment of the present disclosure may provide a system for controlling a flow rate of fluid to a patient through tubing extending from an intravenous bag; said system comprising a drop counter operably engaged with a drip chamber provided between the intravenous bag and the tubing; a roller clamp in electronic communication with the drop counter; wherein the roller clamp includes a bore through which the tubing is received; a roller extending into the bore and contacting the tubing; a drive mechanism operable to push and rotate the roller in one of first direction and a second direction; a processor in communication with the drive mechanism; and a human-machine interface (HMI) functionally linked with the processor. In one embodiment the system may be an automated closed-loop gravity infusion system.

### BRIEF DESCRIPTION OF THE DRAWINGS

Sample embodiments of the present disclosure are set forth in the following description, are shown in the drawings and are particularly and distinctly pointed out and set forth in the appended claims.

FIG. 1 is diagrammatic front elevation view of a gravity infusion system in accordance with the present disclosure;

FIG. 2 is a top, front, right side isometric perspective view of part of an intravenous pole, an intravenous bag, a drip chamber, a drop counter, and a housing assembly of the gravity infusion system of FIG. 1;

FIG. 3 is a fragmentary front elevation view of part of an intravenous bag, drip chamber, and drop counter of the gravity infusion system of FIG. 1;

FIG. 3A is an enlarged top, front, right side isometric perspective view of the drop counter and drip chamber shown in FIG. 3;

FIG. 4 is an exploded top, front, right side isometric perspective view of an inner housing and a roller clamp assembly of the housing assembly, wherein an outer housing of the housing assembly has been omitted for clarity of illustration;

FIG. 4A is a front elevation view of the inner housing and the roller clamp assembly of the housing assembly shown in FIG. 4, wherein the inner housing and roller clamp assembly are shown in an assembled condition;

FIG. 4B is a front elevation/partial section view of the assembled inner housing and roller clamp assembly with some components shown in FIG. 4A omitted therefrom for clarity of illustration;

FIG. 5 is a front elevation view of a second embodiment of the inner housing and roller clamp assembly shown in an assembled condition;

FIG. 5A is a front elevation/partial section view of the assembled inner housing and roller clamp assembly;

FIG. 5B is a left, top, front, isometric perspective view of part of the adjustment assembly of the second embodiment inner housing and roller clamp assembly shown in FIG. 5;

FIG. 5C is a front elevation, partial section view of the assembled inner housing and roller clamp assembly showing the positioning of the first plunger and second plunger as the pin roller moves the roller wheel of the roller clamp relative to the clamp body;

FIG. 5D is a front elevation view of the assembled inner housing and roller clamp assembly showing the tubing movement mechanism contacting the tubing and being positioned to shift the tubing upwardly or downwardly to bring a new section of the tubing into contact with the roller clamp assembly;

FIG. 6 is a flowchart showing a PRIOR ART method of infusing a patient using a gravity IV system; and

FIG. 7 is a flowchart showing an exemplary method of infusing a patient using the gravity infusion system in accordance with the present disclosure.

Similar numbers refer to similar parts throughout the drawings.

### DETAILED DESCRIPTION

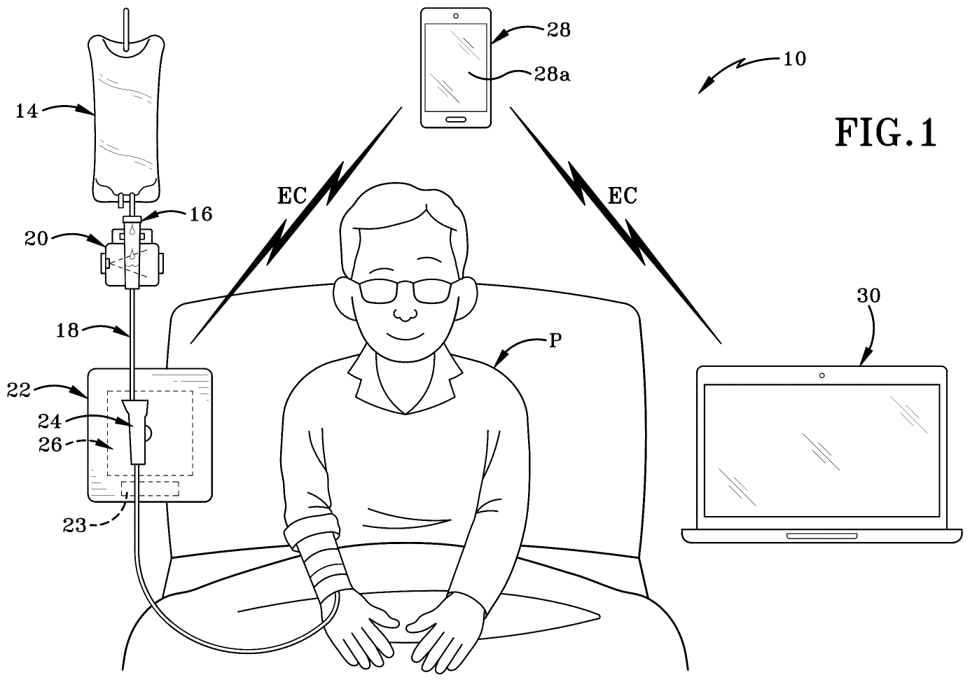

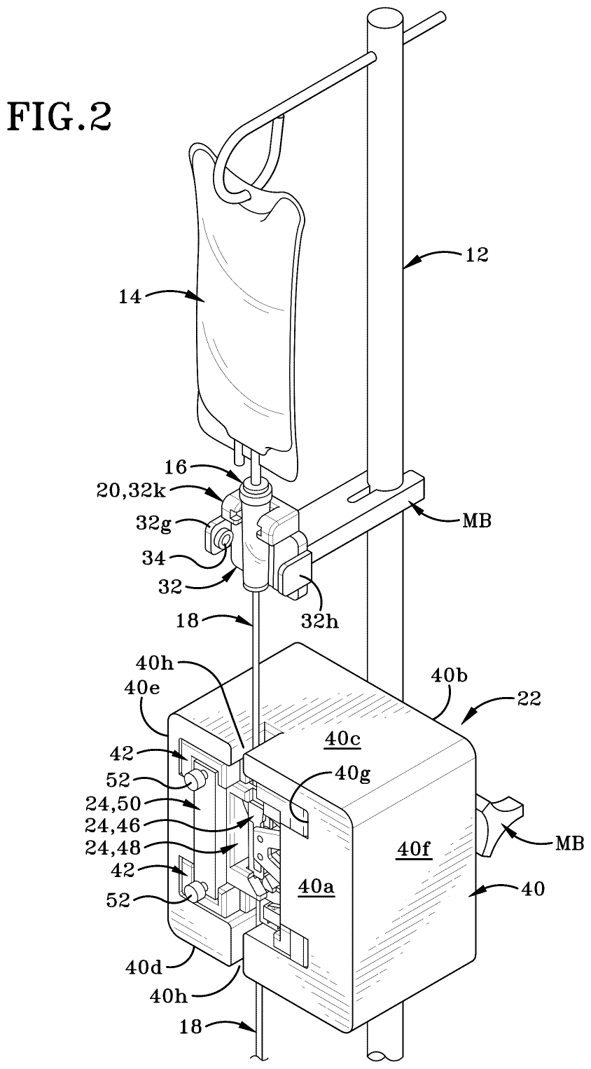

FIGS. 1 to 4B show a diagrammatic representation of a gravity infusion system in accordance with the present disclosure, generally indicated at 10. Gravity infusion system 10 (also referred to herein as “system 10”) includes an intravenous pole 12 (FIG. 2), an intravenous bag 14 suspended from intravenous pole 12, a drip chamber 16 operatively engaged with IV bag 14, and a length of tubing 18 extending outwardly from drip chamber 16. The tubing 18 terminates in an intravascular connector or luer (not shown) which is inserted into the bloodstream of a patient “P” so that the IV bag 14 is placed in fluid communication with the patient's bloodstream. IV bag 14, drip chamber 16, and tubing 18 form part of a fluid source which facilitates delivery of a fluid to a patient's body under force of gravity. Intravenous pole 12 (referred to hereafter as “IV pole 12” or “pole 12”), the intravenous bag 14 (referred to hereafter as “IV bag 14” or “bag 14”), the drip chamber 16, and the tubing 18 with the luer (not shown) are all used in previously-known infusion systems. Since IV pole 12, IV bag 14, drip chamber 16, tubing 18, and the luer attached to the tubing 18 are known in the art, these components will not be described in any particular detail hereafter except as needed.

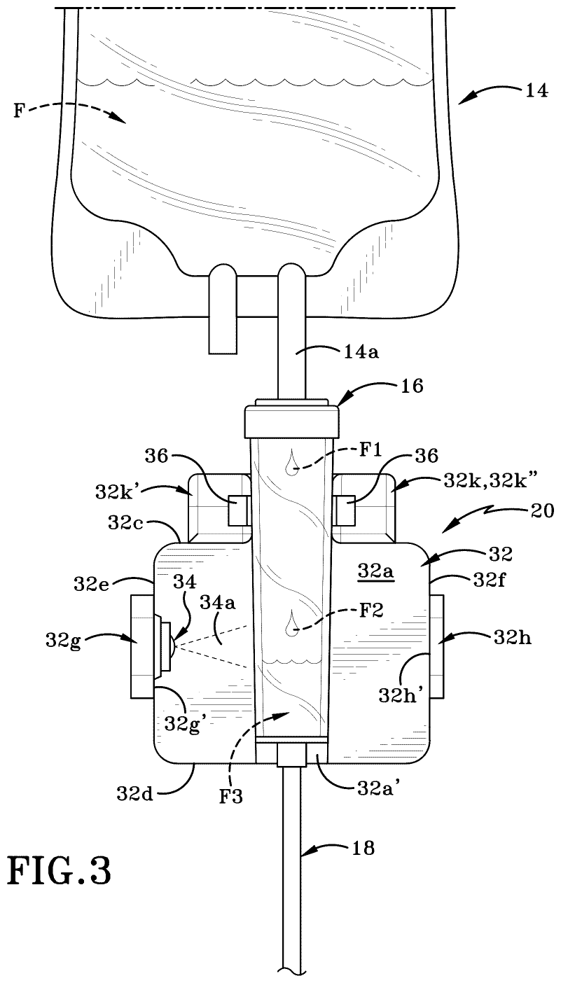

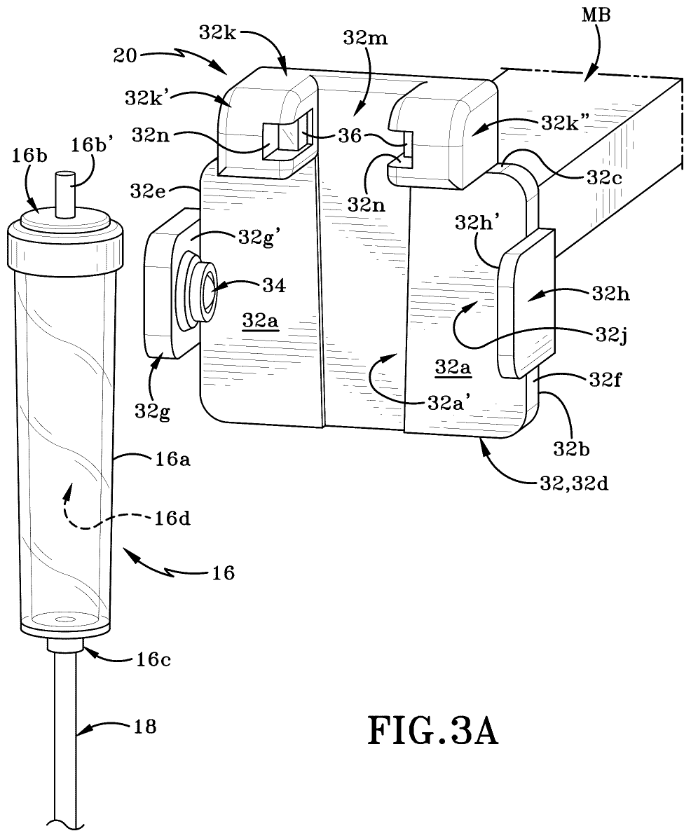

In accordance with an aspect of the present disclosure, a drop counter 20 is mounted on IV pole 12 and is operatively engaged with drip chamber 16 as illustrated in FIGS. 2 and 3. (It will be understood that any suitable drop counter may be used in gravity infusion system 10.) A mounting bracket “MB” is operatively engaged with base 32 of drop counter 20 and mounting bracket “MB” is configured to secure drop counter 20 to IV pole 12 and may of any suitable configuration to position drop counter at a desired location along the length of IV pole 12. Drop counter 20 maybe frictionally engaged with drip chamber 16 in such a way that drop counter 20 is able to able to continuously monitor a rate of fluid drops dripping from the IV bag 14 into the drip chamber 16. In particular, the drop counter 20 counts drops of fluid which drip from IV bag 14 into drip chamber 16 under force of gravity over a period of time. The structure and operation of drop counter 20 will be described in greater detail later herein.

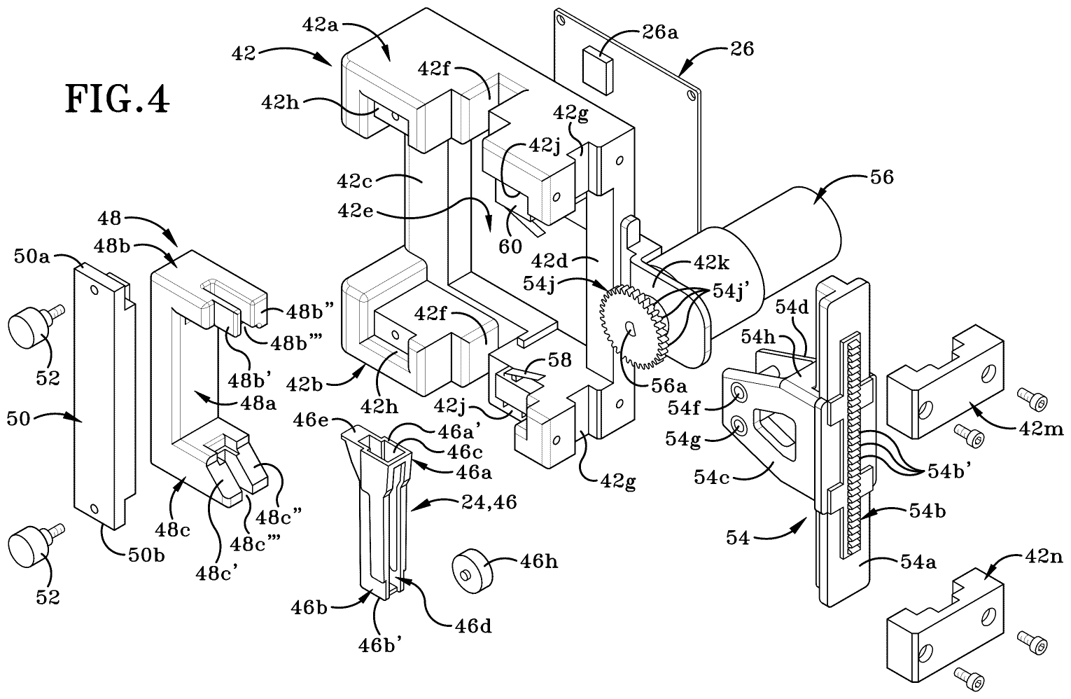

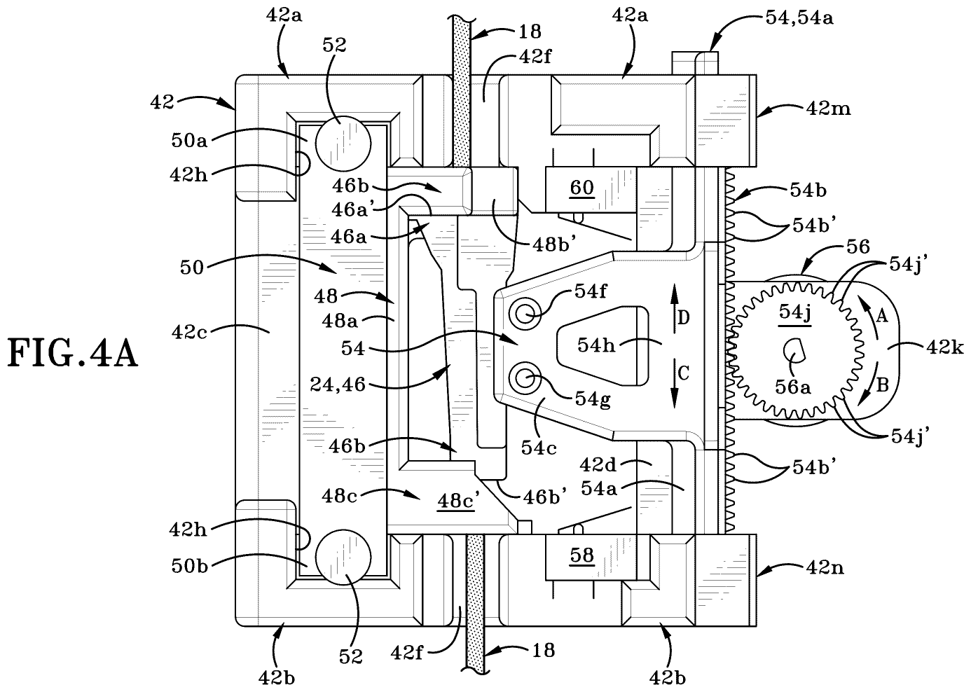

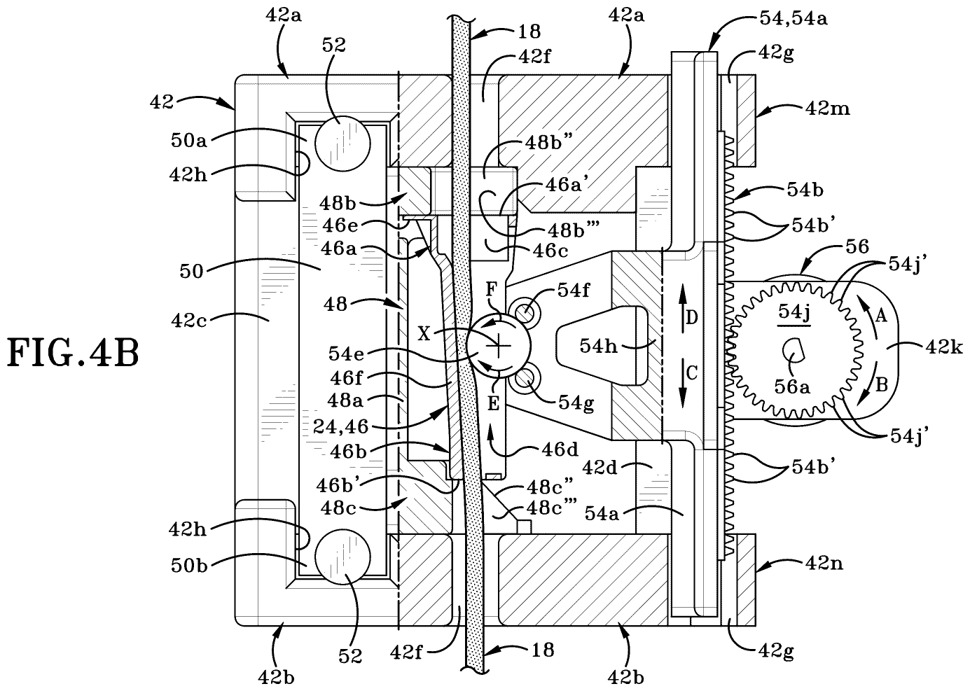

System 10 further includes a housing assembly 22 which is configured to be supported on IV pole 12 a distance below drop counter 20. Housing assembly 22 surrounds and protects, amongst other components, a battery 23 (FIG. 1), a roller clamp assembly 24, and a Printed Circuit Board 26 (FIG. 4). The Printed Circuit Board 26 will hereafter be referred to as “PCB 26”. As shown in FIG. 4, PCB 26 includes a processor 26a (or one or more microprocessors) provided with programming configured to control the functioning of various components of system 10. Battery 23 (FIG. 1) is functionally linked to various electronic components within housing assembly 22 to provide power thereto as needed. (It will be understood that in other embodiments any other suitable power source may be provided in system 10 instead of battery 23.) As will be described later herein, roller clamp assembly 24 comprises a roller clamp 46, a holster 48, and a holder 50. It should be understood that roller clamp 46 maybe any traditional, currently known roller clamp. Holster 48 is a components which is capable of holding any configuration of roller clamp therein and positioning that roller clamp for engagement with and adjustment assembly 54 provided in housing. The presence of holster 48 thereby enables the housing assembly 42 to be a universal component that can receive and be secured to a roller clamp of any configuration.

The processor 26a is functionally linked to drop counter 20, the roller clamp 46 of roller clamp assembly 24 and to an input source 28 for inputting patient data into system 10. As illustrated in the attached figures, the input source 28 maybe a Human-Machine Interface 28 (hereafter “HMI 28”) which may be separate from the intravenous pole 12 and the equipment mounted thereon. Ideally, the HMI 28 is a portable device that may be carried by a practitioner. The HMI 28 is functionally linked to at least some of the rest of system 10 as will be described herein. In other embodiments instead of the HMI 28 being a portable device, the input source may be provided as an integral part of drop counter 20 or an integral part of housing assembly 22 or roller clamp assembly 24. System 10 is designed such that the drop counter 20, housing assembly 22 with roller clamp assembly 24, and the HMI 28 maybe operatively engaged with any currently known IV system which has to be manually operated by a practitioner and turn that currently known IV system to an automated gravity infusion system in accordance with the present disclosure. Patient data may be entered into system 10 via a user interface provided on HMI. In other instances patient data may be stored in a database that is accessed by the HMI in order to retrieve relevant stored patient data for a particular patient.

Drop counter 20 continuously monitors the drip chamber's drip rate and then feeds the drip rate data to processor 26a. The processor 26a analyzes and then controls a drive mechanism of roller clamp 46 of roller clamp assembly 24 to automatically adjust a position of a roller of the roller clamp 46 in order to provide an appropriate and desired dosage of fluid to the patient “P”. The system 10 continuously monitors drip rate and automatically adjusts the roller clamp 46 automatically until the infusion is completed. The structure and functioning of housing assembly 22, roller clamp assembly 24, and PCB 26 and all other components of system 10 will be described in greater detail later herein.

As shown in FIG. 1, system 10 further includes a Human-Machine Interface 28 (hereafter “HMI 28”) which is operatively coupled to PCB 26 to enable a user to enter information about patient “P” and the treatment which is to be provided to patient “P” via system 10. HMI 28 is additionally or optionally operatively coupled to a remote computing system 30. It should be noted that communication between a practitioner and system 10 is achieved through an application uploaded to HMI 28 or via programming provided on the remote centralized computing system 30. The remote centralized computing system 30 is configured to be capable of enabling a practitioner to simultaneous monitor multiple substantially identical infusion systems 10. The structure and operation of HMI 28 and computing system 30 will be described in greater detail later herein.

It will be understood that various components of system 10 are in functionally linked to one another, i.e., are in electronic communication with one another. It will further be understood that one or more of the electronic components of system 10 may include one or more processors provided with programming which controls other components within system 10 and ultimately automatically controls delivery of fluid from IV bag 14 to the patient “P” via tubing 18. In particular, the programming controls the flow rate of fluid from IV bag 14 to the patient “P” via tubing 18. The electronic communication between various components of system 10 maybe arranged in any desired manner which enables programming uploaded into at least one of a processor of drop counter 20, processor 26a of PCB 26, a processor of HMI 28, and a processor of remote computing system 30 to automatically control the delivery of fluid from the IV bag 14 to the patient “P”. FIG. 1 shows two symbols indicated by the reference character “EC” which represents electronic communication between the electronic components of system 10. The electronic communication “EC” symbols are indicative of wireless or wired communication between various components of system 10 and should not be narrowly interpreted as limiting the nature of possible communication between any of the various electronic components of system 10.

Referring now to FIGS. 3 and 3A, drip chamber 16 is illustrated as comprising a transparent vial 16a having an upper end 16b and a lower end 16c. An interior chamber 16d is defined by a circumferential wall of vial 16a. Upper end 16b of vial 16a includes an inlet 16b′ configured to be operatively engaged with a tube 14a (FIG. 3) extending downwardly from IV bag 14. Lower end 16c of vial 16a is configured to be operatively engaged with or integrally formed with tubing 18. FIG. 3 shows that fluid “F” from IV bag 14 drips drop by drop (“F1”, “F2”) into drip chamber 16 via tube 14a and inlet 16b′. A volume of fluid “F3” accumulates in the interior chamber 16d of drip chamber 16 and this fluid “F3” ultimately makes its way into and through tubing 18.

Referring now to FIGS. 2, 3 and 3A, the structure and functioning of drop counter 20 will be described in greater detail. Drop counter 20 comprises a body having a base 32 with a first surface 32a and an opposed second surface 32b. Base 32 further includes a first end 32c, a second surface 32d, a first side 32e, and a second side 32f. First surface 32a and second surface 32b define a first lateral direction between them. First end 32c and second end 32d are opposed and define a longitudinal direction therebetween. First side 32e and second side 32f are opposed and define a second lateral direction therebetween.

As best seen in FIG. 3A, first surface 32a define a generally longitudinally-oriented depression 32a′ therein. Depression 32a′ extends generally from first end 32c of base 32 to second end 32d thereof. Depression 32a′ provides a region of first surface 32a against which drop counter 16 is able to be seated. Depression 32a′ may be slightly tapered moving in a direction from first end 32c towards second end 32d of base 32. The tapering of depression 32a′ is of a shape similar to the tapering of drip chamber 16 which is to be operatively engaged with drop counter 20.

A first arm 32g and a second arm 32h extend outwardly away from first surface 32a of base 32. First arm 32g is located proximate first side 32e and second arm 32h is located proximate second side 32f. As such, second arm 32h is laterally spaced from first arm 32g and extends outwardly from first surface 32a in a same direction as first arm 32g. As shown in FIG. 3, first arm 32g and second arm 32h each have an inner surface 32g′, 32h′, respectively. Inner surfaces 32g′ and 32h′ are opposed to one another. In one embodiment, the inner surfaces 32g′, 32h′ are arranged parallel to one another and a gap 32j (FIG. 3A) is defined between the inner surface 32g′ of first arm 32g and the inner surface 32h′ of second arm 32h.

A U-shaped clamping member 32k is provided on base 32. Clamping member 32k may be provided anywhere on base 32 which enables at least a portion of the clamping member 32k to extend outwardly beyond first surface 32a. As illustrated in FIGS. 2 through 3A, clamping member 32k is located proximate first end 32c of base 32 and extends upwardly and outwardly beyond first end 32c. Clamping member 32k comprises a first jaw 32k′ and a second jaw 32k″ which are laterally spaced from one another and define a channel 32m therebetween. Channel 32m is bounded and defined by a U-shaped inner surface of clamping member 32k defined by inner surfaces of first jaw 32k′ and second jaw 32k″ and part of first surface 32a which spans depression 32a′. Channel 32m is oriented longitudinally, is aligned with an upper region of depression 32a′, and is in fluid communication with gap 32j. Channel 32m is shaped and sized to receive a portion of drip chamber 16 therein. Clamping member 32k may be fabricated such that first jaw 32k′ and second jaw 32k″ flex slightly apart from one another in order to receive drip chamber 16 into channel 32m and then return to their original position to clamp drip chamber 16 between first jaw 32k′ and second jaw 32k″.

A camera 34 (FIGS. 2 through 3A) or any other suitable optical scanner or sensor is provided on one of first arm 32g and second arm 32h of drop counter 20. As illustrated, camera 34 extends into gap 32j from inner surface 32g′ of first arm 32g. It will be understood that camera 34 may instead, be provided on the inner surface 32h′ of second arm 32h and extend into gap 32j from second arm 32h. Camera 34 is operatively engaged with PCB 26 and is controlled by the programming provided in the processor on PCB 26. Camera 34, when actuated by the programming, has a field of view 34a which intersects drip chamber 16 retained in gap 32m of drop counter 20 by clamping member 32k. Camera 34 is actuated to take a photo, video, or other scan or reading of drips of fluid from IV bag 14, such as drips “F1”, “F2”. In particular, camera 34 gathers images or other data relating to drips “F1”, “F2” as the drips drop into the volume of fluid “F3” in drip chamber 16.

Camera 34 provides non-stop visual monitoring of the drip status and is used to report drip status. The data gathered by camera 34 in the form of images is sent electronically to the processor 26a. As will be further discussed, the data may then be sent electronically from PCB 26 to HMI 28 and may further be electronically sent from HMI 28 to remote computing system 30. Data gathered by camera 34 may also be used to report the drip status to a practitioner and initiate alerts and alarms to practitioner.

PRIOR ART systems have no way to monitor backflow and practitioners have to manually put a piece of tape or draw a physical line on the exterior surface of the drip chamber in order to mark a starting point of the infusion. The practitioner will then have to later check the level of fluid in the drip chamber relative to this piece of tape or physical line in order to be detect if there is backflow. If fluid eventually rises above this piece of tape or physical line, then the practitioner will know there is backflow. Currently, manufacturers are working towards providing a physical line on drip chambers to make it easier for practitioners to visually detect backflow.

By contrast, in the currently-disclosed system, monitoring for potential backflow problems is readily addressed by the camera 34 which captures images of the drip chamber 16 to determine drip chamber fluid level and feeding those images to processor 26a. Processor 26a is programmed to continually monitor and compare images to determine if backflow is occurring. The currently-disclosed system will therefore detect possible backflow sooner than would a practitioner who has to continually come around and check fluid levels in the drip chamber 16 for possible problems. Additionally, the currently-disclosed system will automatically issue a visual or audible alert to the practitioner so that corrective action can immediately be taken. The system may furthermore be programmed to automatically stop the infusion if backflow is detected.

As discussed above, camera 34 is used for monitoring and confirming the status of the drip chamber 16 by reading the drip chamber fluid level. A threshold fluid level is programmed into processor 26a in order to set a threshold fluid level for drip chamber 16. Camera 34 acts as a fluid level sensor in that it substantially continuously observes and monitors the actual fluid level within drip chamber 16 and provides associated data to processor 26a. If the actual fluid level rises within drip chamber 16 to a level that is above the predetermined or programmed threshold fluid level, then a backflow situation may exist in drip chamber 16. When this situation arises, an automatic backflow alarm will be triggered in the system. An automatic alert or notification will be issued by the system to a practitioner indicating there is a potential backflow issue in drip chamber 16 which needs to be addressed. The alert may be in the form of an audible alarm emitted by suitable components provided on one or more of drip chamber 16, housing assembly 22, HMI 28, and computing system 30. The alert may additionally or alternatively be a visual alarm in the form of a text message appearing on the screen 28a of HMI 28 and the screen of computing system 30. The alert may additionally or alternatively be a visual alarm in the form of a real-time image of the drip chamber 16 provided by camera 34 and displayed on the display screen 28a of HMI 28 and/or the screen of computing system 30. In particular, the visual image captured by camera 34 will be one that shows the actual fluid level and also indicates the programmed threshold fluid level. The alert provided on screen 28a of HMI 28 and/or the screen of the computing system 30 may include specific instructions for the practitioner to follow in order to resolve the potential backflow issue. As part of those instructions, the practitioner may be directed alerted to physically check on the patient. The instructions provided by the system may also inform the practitioner how to adjust parameters for the delivery of the infusion so as to address the possible backflow situation which triggered the alarm. In other instances, the practitioner may simply follow their training to adjust the parameters for the delivery of the infusion

It should be understood that in some embodiments, drip chamber 16 may be equipped with any suitable type of non-contact fluid level sensor (not shown) which is separate from camera 34 and is used to monitor the actual level of fluid within the drip chamber 16. If the actual fluid level within drip chamber 16 rises above a predetermined (i.e., previously programmed) fluid level, the data provided to processor 26a by the fluid level sensor will trigger a backflow alarm which is identical to the backflow alarms and alerts discussed earlier herein with respect to the use of camera 34 as a fluid level sensor. When such an alarm is triggered by the non-contact fluid level sensor, real time visual feed from the camera 34 may also be provided to the HMI 28 and/or to the computing system 30 to aid the practitioner to determine how best to respond.

As best seen in FIG. 3A, drop counter 20 also includes a pair of infrared sensors 36 mounted in recesses 32n defined in the first jaw 32k′ and second jaw 32k″. Infrared sensors 36 detect the drips “F1”, “F2” when they individually drop into interior cavity 16d of drip chamber 16 and send the gathered information about drips “F1”, “F2” to PCB 26, which in turn may activate camera 34 to take a photograph or gather other data of each individual drip as that drip enters the field of view 34a of camera 34. The drip data gathered by infrared sensors 36 and/or camera 34 is utilized to count the number of drops “F1”, “F2” which enter drip chamber 16 over a pre-determined period of time. The time may be monitored by suitable components provided in drop counter 20 or by programming provided in processor 26a. Drop counter 20 communicates the number of counted drops over the preset time span to processor 26a. In particular, drop counter 20 communicates electronically with processor 26a. Preferably, the counting of the drops by drop counter 20 is accomplished in real time and the electronic communication “EC” with PCB 26 is also accomplished in real time. Instead of the number of counted drops over the preset time span being electronically communicated to PCB 20, that information may additionally or alternatively be communicated electronically to HMI 28 or may additionally or alternatively be communicated electronically to remote computing system 30 if the operation of system 10 is being controlled by HMI 28 or remote computing system 30 instead of being controlled by processor 26a.

It will further be understood that in addition to the camera 34, fluid level sensor, and other sensors described above, the system may additionally be provided with one or more bubble detection sensors (not shown) and associated programming provided in processor 26a. Each bubble detection sensor aids in determining if there are air bubbles trapped in the fluid moving through tubing 18. The sensors may also be able to determine if air is trapped in the fluid within tubing 18. The system will be configured to automatically issue a suitable alert if air bubbles or air is detected in the fluid within tubing 18 and that alert may include audible or visual alarms similar to those issued if backflow is detected. The audible alarm for bubbles or air may be generated in such a way as to sound different to the alarm issued when backflow is detected by the system. The practitioner, when alerted, may then take corrective action to remove the air bubbles or air from fluid within the tubing 18. In addition to generating the automatic alert, the system may be programmed to automatically halt the infusion when bubbles or air are detected in order to give the practitioner time to take appropriate action.

Additionally, one or more pressure sensor (not shown) may be provided in various suitable locations in system 10, such as on drip chamber 16, housing assembly 22, and/or roller clamp assembly 24. Suitable programming will be provided in processor 26a to respond to data provided by the one or more pressure sensors. Each pressure sensor will be operatively engaged with processor 26a and will be configured to detect actual pressure within tubing 18. The programming will use data provided by the pressure sensor to determine if there has been an increase in pressure or decrease in pressure in the tubing 18 relative to a predetermined or preprogrammed pressure. An increase in pressure may signify a potential obstruction or blockage within tubing 18. A decrease in pressure may signify a leak in tubing 18 or in other parts of the system (such as in drip chamber 16). When the pressure rises above a first threshold or drops below a second threshold, the system will be programmed to generate an alert or alarm and automatically halt the infusion in order to give the practitioner time to take appropriate action. Any audible alert or alarm generated by the system may sound different to other alerts or alarms generated by the system for detecting backflow, bubbles, or air. In some embodiments, only one type of audible alarm may be generated for all issues detected by the system and the practitioner will determine the nature of the problem when they view a visual alert on the HMI or physically check on the patient and the various components of the system retained on the IV pole.

As will be discussed later herein, the photographs and/or other drip data gathered through the operation of camera 34 or infrared and/sensors 36 is sent electronically to processor 26a. Processor 26a is provided with programming for utilizing patient data to calculate infusion rates such as the desired total volume of fluid to be infused or the patient's prescriptions. The programming of processor 26a calculates the desired drip rate and then uses this data to determine a desired position of roller clamp 46. Once the desired drip rate has been calculated, the infusion is set up including hanging the IV bag 14 on the IV pole 12, engaging the drop counter 20 with the drip chamber 16, inserting the tubing 18 through the roller clamp 46 and inserting the luer into the patient's body. The infusion process may then be started. When fluid starts dripping from the IV bag 14 into drip chamber 16, the drip detecting and monitoring by drop counter 20 is initiated. As discussed above drop counter 20 measures the drip rate and monitors drip status. Drop counter 20 then feeds the drip data to processor 26a which then utilizes the patient data and drip data to determine the appropriate position of the roller clamp 46. Once the appropriate position of roller clamp 46 has been determined, processor 26a actuates a drive mechanism to move the roller clamp 46 to the determined position in real time. The real time data and drip status can be sent back to the mobile HMI or to the centralized computing system 30 for practitioners.

It should be noted that in some instances the programming which calculates the desired position of roller clamp 46 maybe provided in the HMI 28 instead of the PCB 26. In these instances the gathered data may be sent directly from drop counter 20 to HMI 28 or the data may be sent from PCB 26 to HMI 28. Even if the programming which calculates the desired position of roller clamp 46 is uploaded into the processor 26a, the gathered photographs or other data may still be sent to HMI 28 directly or via PCB 26 for storage or later analysis. The gathered photographs or other data may additionally or alternatively be sent to remote computing system 30 directly from drop counter 20 or from PCB 26 or from HMI 28. The gathered photographs or other drip data may then be stored in a memory of the remote computing system 30 or may be analyzed to better inform an algorithm how to more accurately and reliably control system 10. In some embodiments, the programming which controls delivery of fluid from IV bag 14 to the patient “P” maybe in the remote computing system 30 instead of in a processor provided on drop counter 20 or in processor 26a, or in a processor provided in HMI 28.

Referring now to FIGS. 1, 2, and 4B, housing assembly 22 and roller clamp assembly 24 are shown in greater detail. Housing assembly 22 comprises an outer housing 40 (FIG. 2) which at least partially surrounds and protects an inner housing 42 (FIG. 4). Outer housing 40 includes a first wall 40a and an opposed second wall 40b, a first end 40c and an opposed second end 40d, and a first side 40e and an opposed second side 40f. Outer housing 40 may define an aperture or window 40g (FIG. 2) in first wall 40a through which portions of the inner housing 42 and roller clamp assembly 24 are visible. Each of the first end 40c and second end 40d may define an aperture 40h therein and through which tubing 18 extends when housing assembly 22 is engaged on IV pole 12. Outer housing 40 is provided to surround and protect inner housing 42 and roller clamp assembly 24, as will be later described herein. Outer housing 40 is operatively engaged with IV pole 12 via mounting bracket “MB”, only part of which is visible in FIG. 2. It will be understood that any suitable type of mounting bracket “MB” maybe engaged with outer housing 40 to secure the same to IV pole 12.

Referring to FIGS. 4 through 4B, inner housing 42 and roller clamp assembly 24 are shown in greater detail. In these figures, outer housing 40 is removed for clarity of illustration. Inner housing 42 is configured to be received within an interior cavity (not shown) bounded and defined by first wall 40a, second wall 40b, first end 40c, second end 40d, first side 40e, and second side 40f of outer housing 40. Referring particularly to FIG. 4, inner housing 42 comprises a frame having a first end 42a and a second end 42b which are spaced longitudinally apart from one another. A first support 42c and a second support 42d extend between a region of first end 42a and second end 42b. Inner housing 42 is molded to include various recesses and openings required to accommodate other components of system 10. For example, first end 42a, second end 42b, first support 42c, and second support 42d bound and define an opening 42e in inner housing 42. PCB 26 extends across opening 42e and is secured in place to first and second ends 42a, 42b in any suitable manner. Inner housing 42 further defines channels 42f in each of first end 42a and second end 42b which are aligned with one another and will align with channels 40h defined in outer housing 40 when outer housing 40 is placed around inner housing 42. The purpose of channels 40h, 42f will be described later herein.

Inner housing 42 further defines a slot 42g in a side surface of each of the first end 42a and second end 42b. Slots 42g are aligned with one another also call out the through-hole through which the rack support and rack move in the top end of the inner housing 42. First recesses 42h are defined in a first surface of each of the first end 42a and second end 42b and the first recesses 42h are aligned with one another. Second recesses are also defined in the first surface of each of the first end 42a and second end 42b with the second recesses 42j being laterally spaced from the first recesses 42h. The purpose of each of the slots 42g, first recesses 42h and second recesses 42j will be described later herein. FIG. 4 shows that a flange 42k extends outwardly from second support 42d of inner housing 42. The purpose of flange 42k will be explained further later herein.

Roller clamp assembly 24 is received within a space defined by the various members of the frame of inner housing 42. As indicated earlier herein and referring still to FIG. 4, roller clamp assembly 24 comprises roller clamp 46, holster 48, and holder 50. It should be understood that the specific configuration of roller clamp 46 shown in the attached figures is illustrative of any known type of roller clamp currently on the market. Differently configured roller clamps from those illustrated herein may be utilized in infusion system 10 and the specific configuration of clamp illustrated and described herein should not limit the scope of protection of the present disclosure.

Case 48 is a generally U-shaped component when viewed from a front as in FIGS. 4, 4A and 4B. Holster 48 includes a spine 48a, a first end region 48b, and a second end region 48c. First and second end regions 48b and 48c are located at opposite ends of spine 48a. First end region 48b is generally U-shaped when viewed from above (as in FIG. 4), AND comprises a first arm 48b′ and a second arm 48b″ which are partially separated from one another by a gap 48b′″. First arm 48b′ is shorter in length than is second arm 48b″ B. Gap 48b′″ is located so as to be at least partially aligned with the channels 42f, 40h defined in inner housing 42 and outer housing 40, respectively. Second end region 48c of holster 48 is U-shaped when viewed from above (as in FIG. 4) and includes a first arm 48c′ and a second arm 48c″. A gap 48c′″ is defined between first arm 48c′ and second arm 48c″ and this gap 48c′″ at least partially aligns with gap 48b′″ and channels 42f, 40h defined in inner housing 42 and outer housing 40, respectively.

Roller clamp 46 is configured to be received within and operatively engaged with holster 48 as is illustrated in FIGS. 4A and 4B. FIGS. 4 through 4B show roller clamp 46 includes a body having a first end 46a and a second end 46b. A bore 46c is defined by the body of roller clamp 46 and extends from an opening in first end 46a through to an opening defined in second end 46b thereof. A slot 46d is defined in the body of roller clamp 46. In particular, slot 46d is defined in the side of roller clamp 46 which will be remote from the spine 48a of holster 48 when roller clamp 46 is engaged with holster 48. The slot 46d originates a short distance inwardly from an end surface 46a′ of first end 46a of the body and terminates a short distance inwardly from an end surface 46b′ of second end 46b of the body. Slot 46d is in fluid communication with bore 46c. The purpose of slot 46d will be described later herein.

Body 46 includes a flange 46e which slides under first end 48b of holster 48 when system 10 is assembled and roller clamp 46 is engaged with holster 48. When roller clamp 46 is engaged with holster 48, first end 46a of roller clamp 46 is located beneath a lower surface of first end 48a of holster 48 and second end 46b of roller clamp 46 is at least partially captured within the gap 48c′″ of second end 48c of holster 48. When roller clamp 46 is so engaged within holster 48, bore 46c of roller clamp 46 at least partially aligns with gaps 48b′″ and 48c′″ of holster 48. Bore 46c is thereby is at least partially aligned with channels 42f and 40h in inner housing 42 and outer housing 40, respectively.

At least a portion of the bore 46c defined through roller clamp 46 is bounded and defined by an interior wall 46f (FIG. 4B). Interior wall 46f may be slightly angled relative to a lowermost surface 46b′ of second end 46b of roller clamp 46. When roller clamp 46 is engaged in holster 48, a region of interior wall 46f proximate first end 46a of roller clamp 46 is positioned closer to the spine 48a of holster 48 than is a region of interior wall 46f proximate second end 46b of roller clamp 46. This arrangement of interior wall 46f causes bore 46c to taper in cross section from first end 46a through to second end 46b of roller clamp 46. The reason for this slight incline of interior wall 46f and thereby the tapering of bore 46c will be discussed later herein. In other configurations of roller clamp 46, grooves (not shown) are defined in opposed side surfaces of the roller clamp 46 which extend outwardly from interior wall 46f. The grooves will be in fluid communication with bore 46c and slot 46d. A roller 46h is received at least partially within bore 46c and a portion of the roller 46h protrudes outwardly from the body of roller clamp 46 through slot 46d thereof. FIG. 4 shows the roller 46h exploded away from roller clamp 46. Although not illustrated herein, it should be understood that the circumferential surface of the roller 46h may include knurling since the roller 46h is typically contacted by a human finger in order to push and rotate the roller 46h relative to the body of the roller clamp 46.

When roller clamp 46 is retained within holster 48, tubing 18 is threaded through channel 40h in first end 40c of outer housing 40, through channel 42f defined in first end 42a of inner housing 42, through gap 48b′″ of holster 48, through bore 46c of roller clamp 46, out of gap 48c′″ of cast 48, through channel 42f defined in second end 42b of inner housing 42, and through channel 40h in second end 40d of outer housing 40. When tubing 18 is threaded through the bore 46c of roller clamp 46, the roller 46h thereof contacts the exterior surface of the tubing 18.

Roller clamp 46 is engaged within holster 48 as described above and holster 48, in turn, is retained in position between first and second ends 42a, 42b of inner housing 42 by holder 50. Holder 50 has a first end 50a and a second end 50b. First end 50a and second end 50b are configured to be received within first recesses 42h defined by first and second ends 42a, 42b of inner housing 42. Thumb screws 52 are inserted through holes (not numbered) defined in first end 50a and second end 50b of holder 50 and into holes (not numbered) defined in the portions of first end 42a and second end 42b of inner housing 42 which define first recesses 42h. When thumb screws 52 are installed, holster 48 cannot be disengaged from inner housing 42 and roller clamp 46 is secured within holster 48. If a user wishes to remove or replace roller clamp 46, thumb screws 52 are unscrewed, holder 50 is disengaged from inner housing 42, and holster 48 is then removed from within the space defined between first and second ends 42a, 42b of inner housing 42. Once holster 48 is disengaged from inner housing 42, roller clamp 46 can be disengaged from holster 48.

Referring to FIG. 4, there is shown a drive assembly 54 comprising a rack support 54a with a rack 54b engaged thereon. Rack support 54a is configured to include a first end (unnumbered) which is received within slot 42g defined in first end 42a of inner housing 42 and a second end (unnumbered) which is received within slot 42g defined in second end 42b of inner housing 42. Securement members 42m and 42n are utilized to secure first and second ends of rack support 54a in place within inner housing 42. In particular, rack support 54a is retained within the aligned slots 42g defined in inner housing 42 in such a way that rack support 54a can slide relative to first and second ends 42a, 42b. Rack 54b includes a plurality of teeth 54b′ which are horizontally oriented when rack support 54a is received vertically within slots 42g.

Drive assembly 54 further comprises a pair of laterally-spaced apart legs 54c, 54d (FIG. 4) which extend outwardly beyond a surface of rack support 54a opposite that upon which the rack 54b is provided. Legs 54c, 54d are substantially identically configured relative to one another and are laterally aligned with one another. A pair of pin rollers 54f, 54g extends between opposed inner surfaces of legs 54c, 54d. Each pin roller 54f, 54g comprises a bearing provided in an aperture defined in a respective one of the legs 54c, 54d, and a pin extending between the opposed and aligned bearings. The pins of pin rollers 54f, 54g contact the roller 46h of roller clamp 46 when housing assembly 22 is assembled. Pin rollers 54f, 54g are utilized to move roller clamp 46 in one of a first direction and a second direction when drive assembly 54 is activated (as will be described later herein). A bridge region 54h (FIG. 4) extends between legs 54c, 54d a distance laterally away from pin rollers 54f, 54g, and proximate rack support 54a. When roller clamp 46 and drive assembly 54 are engaged with inner housing 42, an upper surface of bridge 54h is located proximate first end 42a of inner housing 42 and a lower surface of bridge 54h is located proximate second end 42b of inner housing 42.

FIG. 4 through 4B show drive assembly 54 further includes a pinion gear 54j which is mounted on flange 42k extending outwardly from second support 42d of inner housing 42. Pinion gear 54j includes teeth 54j′ which are configured to engage and mesh with teeth 54b′ on rack 54b. A motor 56 is operatively engaged with pinion gear 54j and is operable, via a drive shaft 56a, to rotate pinion gear 54j in a selected one of a first direction “A” (FIG. 4A) and second direction “B”. Rotation of pinion gear 54j in the first direction “A” will cause rack 54b and thereby rack support 54a to slide through slots 42g of first and second ends 42a, 42b of inner housing 42, and move downwardly in a direction “C” relative to first and second ends 42a, 42b. Rotation of the pinion gear 54j in the opposite second direction “B” will cause rack 54b and thereby rack support 54a to move upwardly in a direction “D” through slots 42g and relative to first and second ends 42a, 42b of inner housing 42. As rack support 54a moves downwardly in the direction “C”, roller 46h is caused to rotate in the direction “E” (FIG. 4B) and move downwardly along tubing 18, and thereby decreasing the effective size of the tubing bore 18a. As rack support 54a moves upwardly in the direction “D”, roller 46h is caused to rotate in the direction “F” (FIG. 4B) and move upwardly along tubing 18, thereby increasing the effective size of the tubing bore 18a.

FIGS. 4 through 4B show that a first limit switch 58 and a second limit switch 60 are engaged within second recesses 42j defined by first and second ends 42a, 42b of inner housing 42. First limit switch 58 is configured to be contacted by the lower surface of bridge region 54h extending between legs 54c, 54d when rack support 54a moves downwardly in the direction “C” in response to rotation of pinion gear 54j in the direction “A”. Second limit switch 60 is configured to be contacted by the upper surface of bridge region 54h when rack support 54a moves upwardly in the direction “D” in response to rotation of pinion gear 54j in the direction “B”.

As indicated earlier herein, roller 46h is operatively engaged with legs 54c, 54d and is rotatably urged towards engagement with roller clamp 46 by pin rollers 54f, 54g. Pin rollers 54f, 54g help to ensure that roller 46h is able to rotate smoothly about an axis “X” (FIG. 4B) in either of directions “E” and “F”. Since tubing 18 abuts interior wall 46f of roller clamp 46, legs 54c, 54d and the pin rollers 54f, 54g provided thereon tend to urge roller 46h into contact with tubing 18. When rack support 54a and thereby legs 54c, 54d move downwardly in the direction “C” in response to rotation of gear 54j in the direction “A”, the inclined angle of interior wall 46f causes roller 46h to compress tubing 18 and thereby narrow the bore 18a (FIG. 4B) therethrough. The narrowing of bore 18a causes a decrease in a flow rate of fluid “F3” moving from drip chamber 16 through tubing 18 towards the luer at the patient-end of the tubing 18. Continuing movement of rack support 54a in the direction “C” could ultimately completely halt all flow of fluid through bore 18a of tubing 18. When limit switch 58 is contacted by bridge 54h of drive assembly 54, power from motor 56 is cut and rotation of pinion gear 54j in the direction “A” is halted. Consequently, continued movement of rack support 54a in the direction “C” is halted.

Furthermore, when rack support 54a and thereby legs 54c, 54d of adjustment assembly move in the direction “D” in response to rotation of pinion gear 54j in the direction “B”, the inclined angle of interior wall 46f of clamp causes roller 46h to compress tubing 18 to a lesser extent. Consequently, the flow rate of fluid “F3” through bore 18a of tubing 18 from drip chamber 16 tends to increase. Continued movement of rack support 54a in the direction “D” brings bridge 54h of drive assembly 54 into contact with limit switch 60. When this occurs, power from motor 56 to pinion gear 54j is cut and rotation of pinion gear 54j in the direction “B” is halted. When rotation of pinion gear 54j ceases then movement of the rack support 54a in the direction “D” is also halted.

A practitioner is able to set and/or adjust the desired flow rate of fluid “F3” through tubing 18 from drip chamber 16 for any particular patient “P” utilizing HMI 28 (FIG. 1). Once the patient data (including treatment data) is entered into HMI 28, the system will automatically actuate and deliver the appropriate dosage of fluid to the patient “P”. HMI 28 is an electronic device such as a smartphone, tablet, or any other type of hand-held computing system which is capable of communicating with processor 26a (FIG. 4) on PCB 26 as indicated at “EC” in FIG. 1. The communicating “EC” is preferably accomplished wirelessly but in other instances the communicating “EC” maybe accomplished via hard-wiring. One or both of HMI 28 and processor 26a is provided with programming configured to control the various components of system 10. For example, an application or other programming configured to control system 10 maybe downloaded to HMI 28. A medical practitioner will then enter patient data and treatment data into the application or programming using the user interface 28a on HMI 28. The patient data may include information such as the fluid dosage to be delivered to the patient via system 10. The entered patient data will be used to set a flow rate of fluid “F3” to be delivered to patient “P” in order for an appropriate dosage of fluid to be delivered to the patient.

Once the user has set the flow rate for patient “P” on the HMI 28 the information will be communicated “EC” to processor 26a on PCB 26. Programming provided in processor 26a (or programming in HMI 28 accessible to processor 26a via connection “EC”) will actuate drop counter 20 to count the number of drops such as “F1”, “F2” (FIG. 2) dripping in drip chamber 16 over a preset period of time. The number of drops counted by drop counter 20 will be communicated wirelessly to processor 26a. Programming in processor 26a or HMI 28 (or programming in computing system 30) will actuate motor 56 as needed to set the position of roller 46h relative to roller clamp 46 to obtain the desired flow rate at the luer on the patient-end of the tubing 18. If the flow rate of fluid through tubing 18 has to be decreased, motor 56 will rotate the gear 54j in the direction “A” (FIG. 4B). As described earlier herein, when gear 54j rotates in the direction “A” the rack support 54a is moved in the direction “C”. Roller 46h will thereby be caused to move downwardly in the direction “C” along the inclined interior wall 46f of roller clamp 46. As roller 46h moves in the direction “C”, roller 46h compresses tubing 18 and slows the flow rate of fluid through tubing 18. Motor 56 will rotate gear 54j in the direction “A” to a sufficient degree, based on the programming, to cause the desired flow rate of fluid through tubing. Once the desired flow rate is achieved, motor 56 will cease rotating gear 54j and thus the position of roller 46h relative to roller clamp 46 will be fixed and the flow rate through tubing 18 will be maintained.

If the practitioner at a later time decides the flow rate of fluid through tubing 18 needs to be increased or decreased, he or she will manipulate the user interface 28a on HMI 28 to adjust the flow rate accordingly. A signal is communicated “EC” from HMI 28 to PCB 26. PCB 26 will, in turn, actuate motor 56 to rotate in the necessary direction “A” or “B” to change the flow rate. For example, if it is decided by the practitioner to decrease the flow rate of fluid through tubing 18, the PCB 26 will actuate motor 56 to rotate the gear 54j in the direction “A”. Rotation of gear 56j in the direction “A” will, in turn, cause rack support 54a to move in the direction “C”. Movement of rack 54a in the direction “C” will cause roller 46h to rotate in the direction “E” and move along interior wall 46f of clamp in the direction “C”, thereby increasing the compression of tubing 18 by roller 46h, thus effectively decreasing the size of the bore 18a of the tubing 18. As a consequence, the flow rate of fluid through tubing 18 will decrease. Alternatively, if it is decided by the practitioner to increase the flow rate of fluid through tubing 18, the PCB 26 will actuate motor 56 to rotate the gear 54j in the direction “B”. Rotation of gear 56j in the direction “B” will, in turn, cause rack support 54a to move in the direction “D”. Movement of rack 54a in the direction “D” will cause roller 46h to rotate in the direction “F” and move along interior wall 46f of clamp in the direction “D”, thereby decreasing the compression of tubing 18 by roller 46h, thus effectively increasing the size of the bore 18a of the tubing 18. As a consequence, the flow rate of fluid through tubing 18 will increase. When the preset desired flow rate is achieved, a signal from PCB 26 to motor 56 will switch motor 56 off. Consequently, the position of roller 46h relative to roller clamp 46 will be maintained and therefore the flow rate through tubing 18 will be maintained.

The programming enables a constant monitoring of the information provided by drop counter 20 in real time. The programming further enables real time adjustment of the positioning of the roller clamp 46 by selective activation of motor 56 and selective rotation of gear 54j in one or the other direction “A” or “B” as needed. If drop counter 20 detects a faster rate of drops “F1”, “F2” from IV bag 14 into drip chamber 16, then a signal from the PCB 26 to motor 56 will be automatically sent and motor 56 will be actuated to rotate gear 54j in the direction needed to ensure the preset flow rate from tubing 18 is maintained. Actuation of motor 56 will result in a change in the position of roller 46h relative to roller clamp 46 to adjust the flow rate through tubing 18 as needed. Once the roller 46h has been moved to a new relative position, motor 56 will be halted and the flow rate through tubing will be maintained at the preset rate. It should be noted that after initial entry into HMI 28 of patient data and a desired flow rate of the fluid from IV bag 14, system 10 will automatically monitor flow rate and make adjustments in the position of roller clamp 46 to meet the entered desired flow rate.

When no further drops of fluid are counted by drop counter 20 because IV bag 14 is essentially empty or some other issue has arisen with IV bag, that information will be electronically communicated “EC” to PCB 26 (or HMI 28 or remote computing system 30) and the system 10 will automatically issue an alarm so that a practitioner is summoned. The alarm may be a visible or audible alarm or the alarm may take the form of an electronic communication such as a text message, automated voice message etc.

It will be understood that HMI 28 maybe utilized to monitor and automatically control several fluid delivery systems for a plurality of different patients. Once the patient data and desired fluid flow rate for each patient has been entered into HMI 28 via the user interface 28a, the programming of HMI 28 or the PCB 26 of each of the different systems 10 will automatically monitor and control delivery of the fluid to the patient connected to that particular system. A single practitioner can therefore be responsible for fluid delivery to multiple patients in real time.

Remote computing system 30 may form part of a single system 10 or may be electronically connected to multiple identical systems. Remote computing system 30 may be utilized to provide programming to the various systems to control operation of the components thereof. Alternatively or additionally remote computing system 30 may be used to store information about patients and their desired treatment with fluids. Alternatively or additionally remote computing system 30 may include programming to analyze data gathered by drop counters, positioning of clamps etc. to better enable the systems to accurately and reliably deliver a desired flow rate of fluid to patients. Remote computing system 30 may also be utilized to monitor and direct multiple practitioners in different locations at substantially the same time.

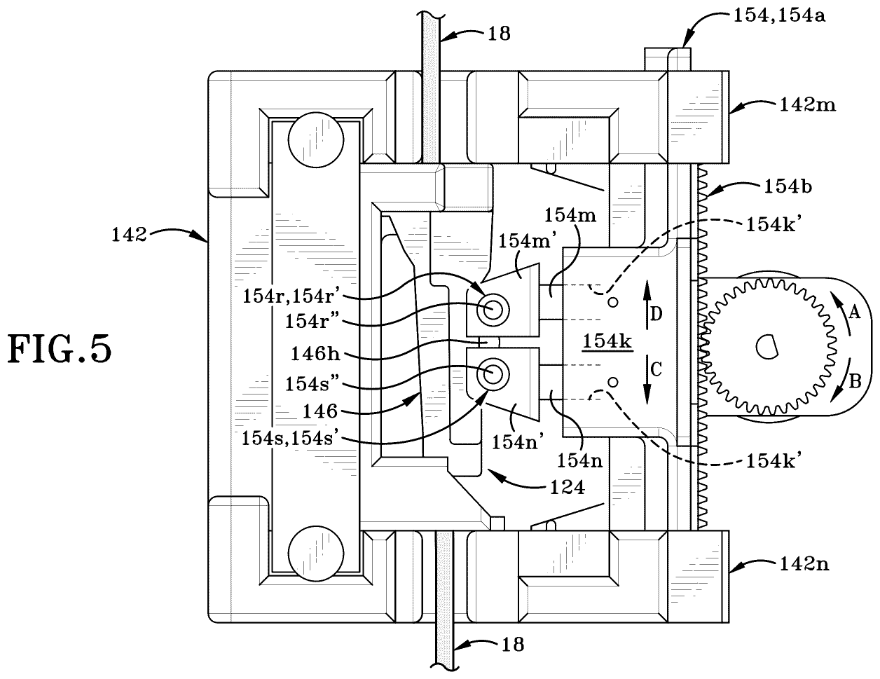

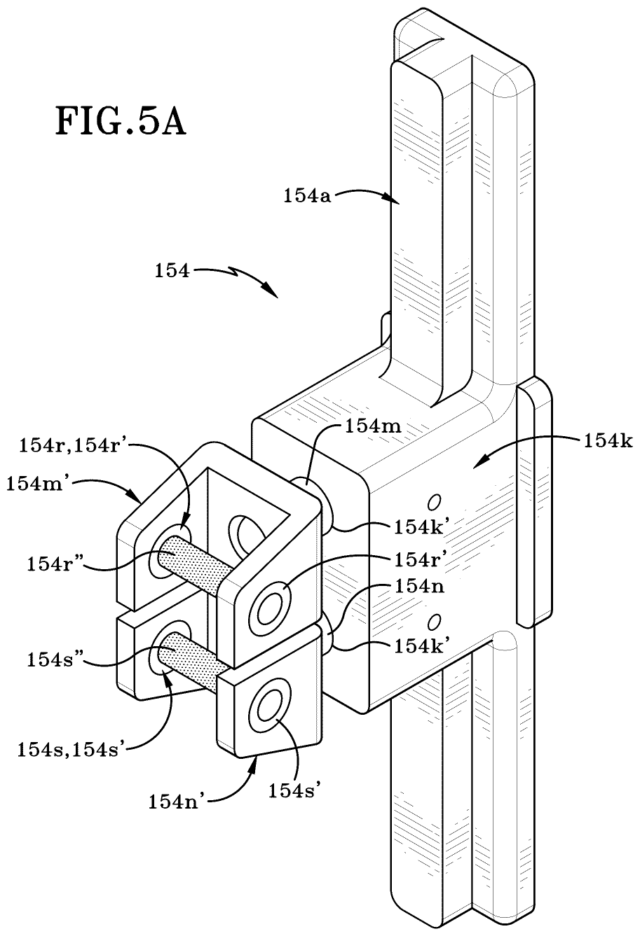

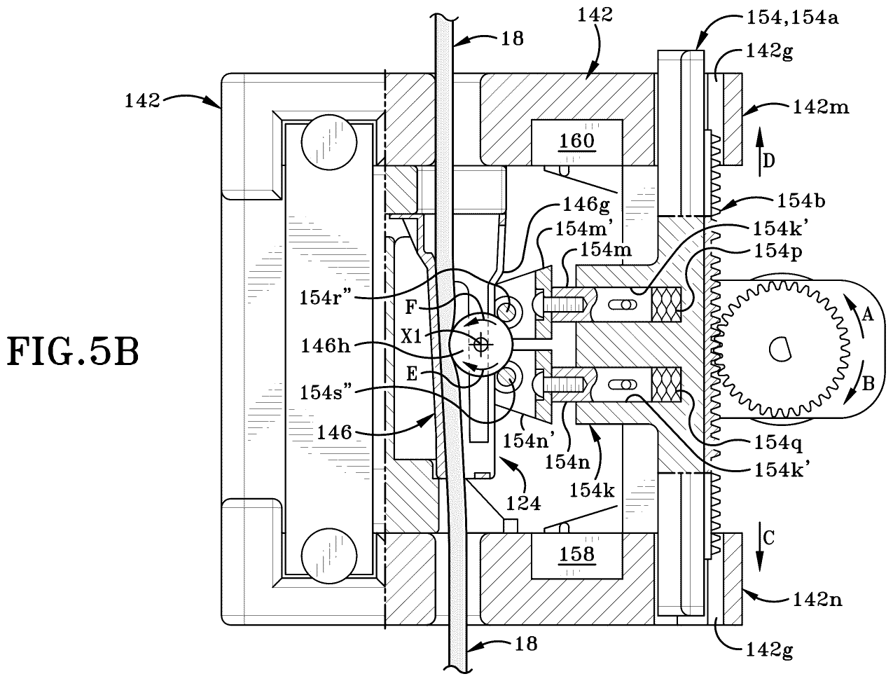

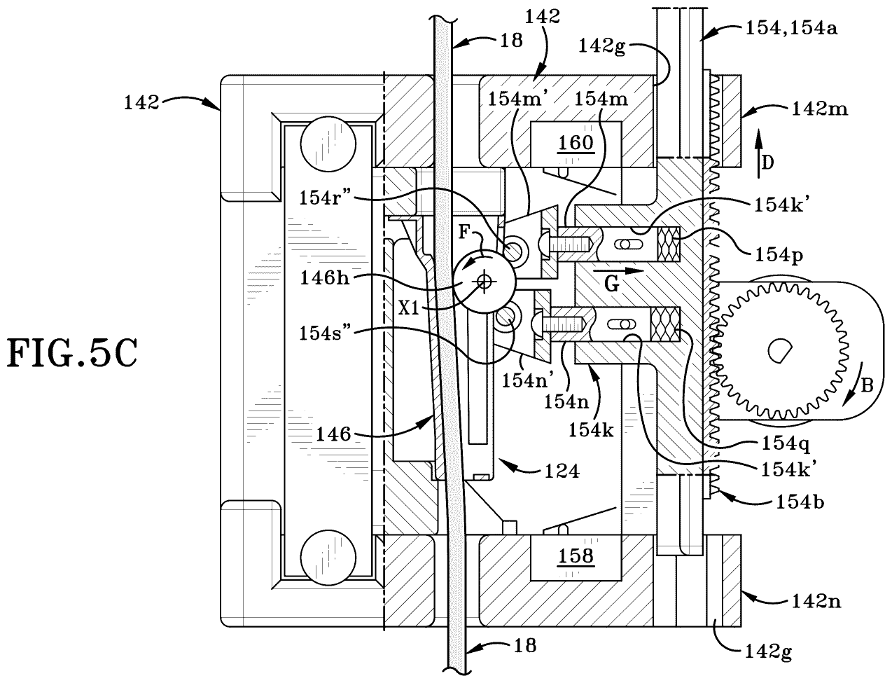

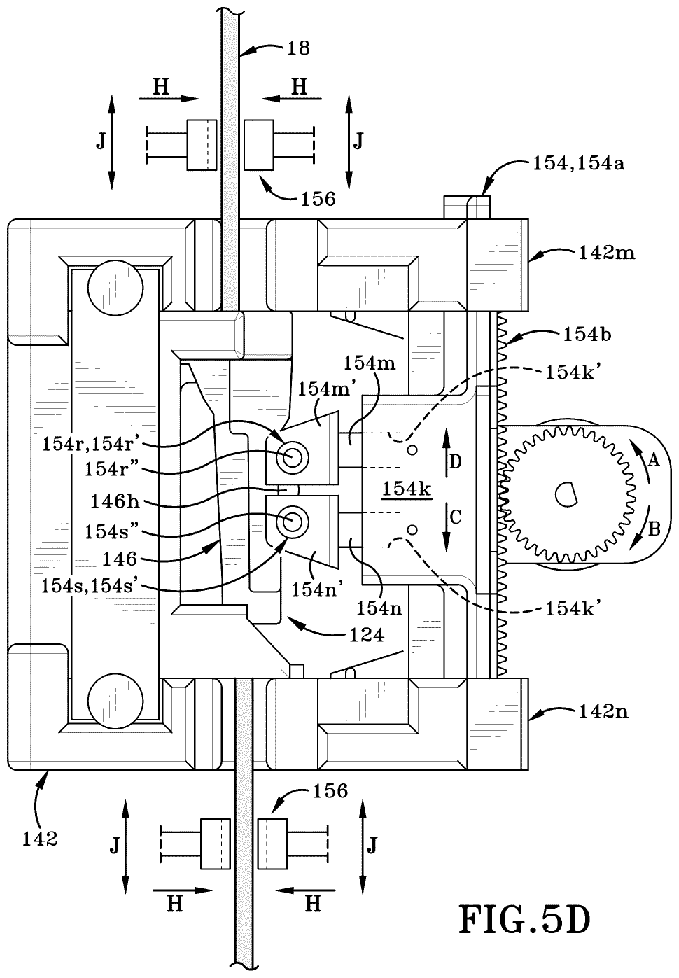

Referring now to FIGS. 5 through 5D, there is shown a second embodiment of part of a housing assembly for use in system 10 in accordance with the present disclosure. In particular FIGS. 5 through 5D show an inner housing 142, a roller clamp assembly 124, and an adjustment assembly 154 in accordance with the present disclosure. (Although not illustrated in these figures, it should be understood that the housing assembly of FIGS. 5 through 5D also includes a processor similar to processor 26a shown in FIG. 4). Inner housing 142 is identical in structure and function to inner housing 42 and therefore will not be described in any additional detail herein. Similarly, roller clamp assembly 124 is identical in structure and function to roller clamp assembly 24. Roller clamp assembly 124 will not be described in any additional detail herein other than to reiterate that the holster which forms part of the housing assembly is a universal holster which is able to receive any configuration of roller clamp therein and serves to secure the various roller clamps in place within the housing assembly in a position where a roller wheel of the held roller clamp is able to be contacted ad adjusted by adjustment assembly 154. Adjustment assembly 154 has many components which are identical in structure and function to drive assembly 54 and these components will therefore not be described in any additional detail herein.

Referring still to FIGS. 5 through 5D, adjustment assembly 154 comprises a rack support 154a with a rack 154b provided thereon. Rack support 154a is configured to include a first end (unnumbered) which is received within a slot 142g (FIG. 5A) defined in a first end of inner housing 142 and a second end (unnumbered) which is received within an opposed slot 142g defined in a second end of inner housing 142. Rack support 154a is engaged within inner housing 142 in an identical manner to how rack support 54a is engaged within inner housing 42. Securement members 142m and 142n are utilized to secure first and second ends of rack support 154a in place within inner housing 142 in the same way as securements 42m and 42n secure rack support 54 to inner housing. Rack 154b includes a plurality of teeth 154b′ which are horizontally oriented when the housing assembly is engaged on an IV support and is positioned beneath IV bag 14 as illustrated in FIG. 1. In particular, the plurality of teeth 154b′ are horizontally oriented when rack support 154a is received vertically within slots 142g of inner housing 142. Rack 154b, its structure and function, are identical to rack 54b and therefore will not be described in any further detail herein.

Adjustment assembly 154 differs from adjustment assembly 54 in a number of aspects which will be described hereafter.

Firstly, instead of a pair of laterally-spaced apart legs 54c, 54d being provided on rack support 54, adjustment assembly 154 includes a differently configured adjustment housing 154k. The adjustment housing 154k extends outwardly from the surface of rack support 154a which is opposite the surface upon which rack 154b is provided. Adjustment housing 154k extends away from rack support 154a and towards roller clamp 146. It will be understood that the roller clamp 146 and roller 146h thereof are substantially identical in structure and function to roller clamp 46 and roller 46h and therefore will not be described herein in any further detail. It will, of course be understood that the specific configuration of roller clamps 46, 146 and rollers 46h, 146h illustrated herein are exemplary only since the holster 48, 148 provided in the housing assembly is configured to receive and secure any one of a variety of differently configured roller clamps therein.

As best seen in FIGS. 5B and 5C, adjustment housing 154k defines a pair of slots 154k′ therein which originate in openings defined in the surface of adjustment housing 154k which is opposite roller clamp 46. A first plunger 154m is provided within a first of the two slots 154k′ and a second plunger 154m is provided within a second of the two slots 154k′. The two slots 154k′ and therefore the first and second plungers 154m, 154n are vertically spaced apart from one another and are aligned with one another. A first coil spring 154p is provided within the first slot 154k′ which receives first plunger 154m and urges the first plunger 154m towards roller clamp 146. Similarly, a second coil spring 154q is provided within the second slot 154k′ and urges the second plunger 154n towards roller clamp 146. A first end cap 154m′ is provided on the free end of first plunger 154m and a second end cap 154n′ is provided on the free end of second plunger 154n.

Each of the first end cap 154m′ and second end cap 154n′ is U-shaped when adjustment assembly 154 is viewed from above as in FIG. 5D. A first bearing assembly 154r is provided on first end cap 154m′ and a second bearing assembly 154s is provided on second end cap 154n′ Each of the first bearing assembly 154r and second bearing assembly 154s comprises aligned bearings seated in apertures defined in the opposed legs of the respective U-shaped cap and a pin extending between the bearings and spanning the distance between the bearings. In particular, first cap 154m′ includes bearings 154r′ and a pin roller 154r″. Second end cap 154n′ includes bearings 154s′ and a pin roller 154s″. Each pin roller 154r″ and 154s″ is encased in a resilient exterior layer. The resilient layer is best seen in FIG. 5A. In one embodiment, the resilient exterior layer is comprised of rubber. The rubber is provided to improve the ability of the respective pin roller 154r″ and 154s″ to grip the circumferential surface of the roller 146. As can be seen from FIGS. 5 to 5D, pin roller 154r″ and 154s″ contact spaced apart locations on the surface of roller 146h. In particular, the pin rollers 154r″ and 154s″ contact spaced apart locations on the circumferential surface of the roller 146h. It should be noted that the distance between the legs of the U-shaped first end cap 154m′ and second end cap 154n′ is slightly greater that the width of the roller 146h on roller clamp 146. When adjustment assembly 154k is brought into contact with roller 146h, a portion of the circumferential surface of roller 146h is received in the gap between the two legs of the first end cap 154m′ and second end cap 154n′ and contacts the resilient exterior surfaces of pins 154r″ and 154s″.

Springs 154p, 154q urge first and second plungers 154m, 154n into operative engagement with roller 146h and in turn urge roller 146h towards roller clamp 146. Springs 154p, 154q allow a contact point between plungers 154m, 154n and roller 146h to move vertically relative to roller clamp 146h in order to accommodate different external profiles of variously-configured roller clamps. When a differently-configured roller clamp 146 is engaged with the housing assembly, the first plunger 154m and second plunger 154n may be adjusted to extend further outwardly from or less father outwardly from adjustment assembly 154. The adjustment of first plunger 154m and second plunger 154n, in turn, adjusts the relative positions of first pin roller 154r″ and second pin roller 154s″. The resilient exterior surfaces of pins 154r″ and 154s″ directly contact roller 146h and ensure that roller 146h is able to easily rotate about axis “X1” (FIG. 5A) as the adjustment assembly 154 moves upwardly in the direction of arrow “D” or downwardly in the direction of arrow “C”. The direct contact between pins 154r″ and 154s″ and roller 146h also pushes (or carries) roller 146h upwardly or downwardly as the adjustment assembly 154 moves in the direction “D” or “C”. As adjustment assembly 154 and roller 146h move upwardly or downwardly relative to roller clamp 146, the first plunger 154m and second plunger 154n may experience different forces thereon as roller 146h travels relative to the roller clamp 146. Springs 154p, 154q are able to be compressed slightly inwardly towards rack 154b and then return to their original uncompressed state as these different forces are experienced. FIG. 5C, for example, shows first end cap 154m on first plunger 154m being moved inwardly towards rack 154b in the direction indicated by arrow “G” as force applied to first plunger 154m by its contact with roller wheel 146h slightly compresses the associated spring 154p. It will be understood that when that force is released when adjustment assembly 154 is moved in an opposite direction to arrow “D”, then spring 154p will return to its uncompressed state shown in FIG. 5B. Because springs 154p, 154q are able to be compressed based on the forces the first plunger 154m and second plunger 154n experience, the contact between first and second plungers 154m, 154n and roller wheel 146h more closely mimics the light but firm touch a human finger will apply to roller wheel 146h to push and rotate the same than if the spring-loaded plunger mechanism was not utilized.

As roller 146h is rotated and pushed upwardly towards the first end 146a of roller clamp 146 by first plunger 154m and second plunger 154n, the roller 146h moves further away from tubing 18 and fluid is able to flow more freely through tubing 18. As roller 146h is rotated and pushed downwardly towards the second end 146b of roller clamp 146 by first plunger 154m and second plunger 154n, the roller 146h moves closer towards tubing 18, deforming the same and reducing the rate of fluid flow through tubing 18. The engagement of adjustment assembly 154 with its two independently operable plungers 154m, 154n closely mimics the soft but firm touch of a human finger operating roller 146h. This type of push and rotate manipulation of roller 146h enables precision adjustment of the position of roller 146h relative to roller clamp 146 and therefore more precise control of the fluid flow rate through tubing 18 than was possible with systems which use roller clamps operated on their own by way of human touch.

Limit switches 158, 160 are provided to limit the travel of adjustment assembly 154 in the same manner as limit switches 58, 60 limit the travel of adjustment assembly 54. The limit switches 158, 160 also help the processor to determine the desired and actual location of roller 146h. A strain gauge (not shown) may be provided in the system to measure the force on first plunger 154m and second plunger 154n in order to better map the forces in the system and find the roller position. The intent of the disclosed system is to be able to control roller 146h like a human finger and prevent over-stressing of roller 146h and roller clamp 146.

Machine operation of roller 146h may easily result in overuse of roller 146h. Therefore to aid in preventing overuse of the roller clamp 146, the system needs to count how many times the roller 146h is rolled upwardly or downwardly relative to roller clamp 146. It is not contemplated that the roller 146h will continuously be in motion. The housing assembly, shown in FIGS. 5 through 5D, may therefore be provided with a dedicated counter which counts the number of times adjustment assembly 154 is used to position roller 146h. This counter may be provided at any suitable location within the housing assembly including as a programming function provided on the processor or a physical counter mounted on physical components within the housing assembly. Optical sensors or other types of sensors may form part of the counter or may be utilized in conjunction with the counter. If the counted number of times the roller is rolled by the counter exceeds a predetermined threshold, the processor determines that the roller clamp 146 is malfunctioning or that there is some other type of issue with the IV set. In these instances, the HMI may send an error message to the practitioner monitoring the system or sound an appropriate alert or alarm.

Another issue which periodically occurs with gravity IV systems is that tubing may become deformed over time because of roller contact therewith. This deformation is known as creep. In PRIOR ART gravity IV sets, the practitioner monitoring the patient will release the roller clamp and slide the tubing relative thereto until the roller clamp is positioned adjacent a length of tubing which not experiencing deformation. The gravity IV systems disclosed herein are contemplated to include a mechanism for temporarily disengaging the roller 46h, 146h from the tubing and then sliding or jogging the tubing relative to the respective housing assembly to move an un-deformed length of tubing into the roller clamp bore 146d. For example, FIG. 5D shows a tubing movement mechanism or “jogging mechanism” 156 which is utilized to shift the tubing 18 if the section of the tubing engaged in the roller clamp assembly becomes deformed. The tubing movement mechanism 156 moves in the direction indicated by arrows “H” in FIG. 5D and engages or grasps the tubing 18 above and below roller clamp 146. Arrows “J” indicate possible movements of the tubing 18 relative to the inner housing 142 when jogging mechanism 156 is activated. When it is determined the tubing 18 is experiencing creep or deformation, the roller 146h will be moved by the adjustment assembly 154 to a location where the roller 146h has minimal to no contact with the tubing. The jogging mechanism 156 will be activated to grasp the tubing 18 and to pull a length of tubing 18 upwardly or downwardly relative to the roller clamp 146, as indicated by arrows “J”, and so that an un-deformed section of tubing 18 is located within the bore of the roller clamp 146. The jogging mechanism 156 will then release the tubing 18 (moving in the opposite direction to arrows “H”, the adjustment assembly 154 will be activated, and the roller 146h will be moved back into engagement with the tubing 18 within roller clamp 146. This jogging process will be controlled via programming in the processor.

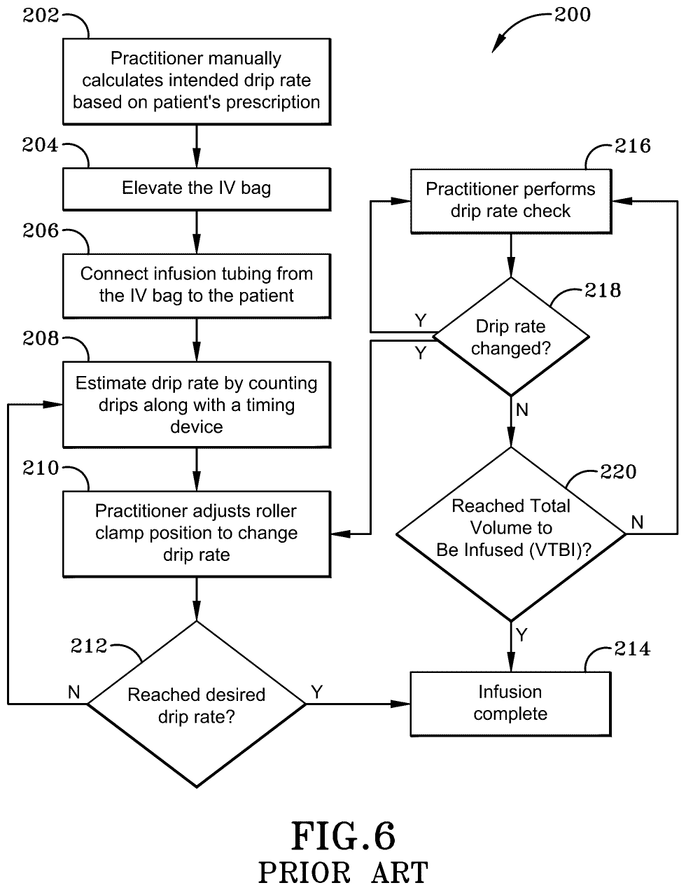

Referring now to FIG. 6, there is shown a PRIOR ART method of infusing a patient, generally indicated at 200. In a first step 202 of the PRIOR ART method a medical practitioner manually calculates a drip rate for the infusion based on a patient's prescription. In step 204 the practitioner will elevate an IV bag containing a fluid to be delivered to the patient on an IV pole. A drip chamber is positioned below the IV bag and a roller clamp will be engaged with tubing extending from the drip chamber. In step 206, the practitioner will connect the IV bag to the patient by inserting a luer provided on the free end of the tubing extending from the drip chamber and IV bag. In step 208, the practitioner will estimate the drip rate of fluid being delivered to the patient by counting drops of fluid falling from the IV bag into the drip chamber over a period of time which is measured with a timing device such as a watch. Using the information determined in step 208 the practitioner will manually adjust the roller clamp position to change the drip rate to try and match the drip rate calculated in step 202. The manual adjustment of the roller clamp position is indicated at 210. In a step 212, the practitioner will determine if the desired drip rate has been achieved. If the answer to the question in step 212 is “No” then the practitioner will return to step 208 and estimate the drip rate again by manually counting drops dripping into the drip chamber and then repeat steps 210 and 212. When the answer to the question of step 212 is “Yes” then in some rare instances the infusion will continue until all fluid from the IV bag has been delivered to the patient and the infusion is completed as at step 214. In most medical settings when the answer to the question of step 212 is “Yes” then a period of time will be allowed to pass and in a step 216 the practitioner will perform a drip rate check by once again by observing and manually counting drips falling from the IV bag into the drip chamber over a period of time. In a next step 218, the practitioner will compare the observed drip rate from step 216 with the drip rate of step 212 to determine if the actual drip rate has changed. If the answer to that question is “Yes”, i.e., the drip rate has changed, then steps 210 and 212 are repeated i.e., the practitioner will manually adjust the roller clamp position to change the drip rate to try and match the drip rate calculated in step 202 and will then determine if the desired drip rate has been reached. Another period of time will be allowed to pass and then steps 216 and 218 are repeated.

If the answer to the question of step 218 whether the drip rate has changed is “No”, then a period of time is allowed to pass and in a step 220 the practitioner determines if the total Volume to Be Infused (VTBI) has been reached. If the answer to the question of step 220 is “Yes” then the infusion is complete, as in step 214.

As is evident from the description of the PRIOR ART method 200 above, this method of infusion is time consuming and relies heavily on the practitioner being accurate in their calculations, observations, and decisions. PRIOR ART method 200 therefore includes the possibility for human error to creep into the infusion process.

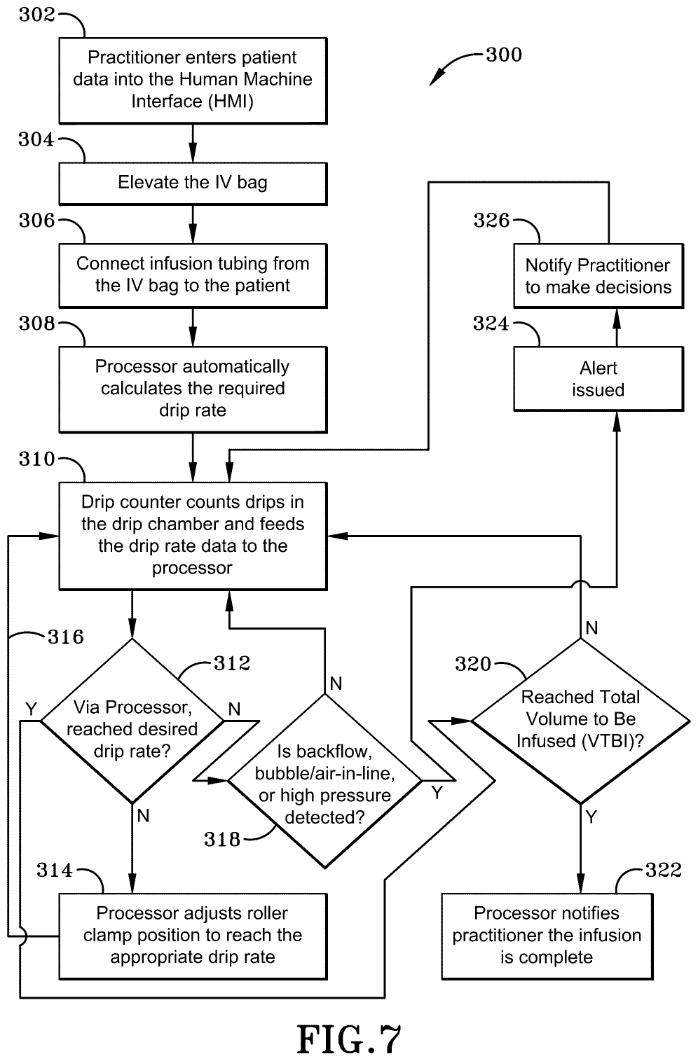

Referring now to FIGS. 7 and 1, the method of infusion a patient using gravity infusion system 10 of the present disclosure is illustrated and generally indicated at 300. Method 300 includes a first step 302 in which the practitioner enters patient data into the Human-Machine Interface (HMI) 28 (FIG. 1). The patient data entered into HMI 28 includes, amongst other information, the patient's prescription for infusion of a fluid. Alternatively, although not shown in FIG. 7, the HMI 28 accesses a database in which patient information, such as patient prescriptions, is stored, and retrieves patient data relevant to a particular patient to be treated.

In step 304, the practitioner elevates an IV bag 14 containing a fluid to be delivered to the patient on an IV pole 12. A drip chamber 16 is positioned below the IV bag 14 and a drop counter 20 is engaged with the drip chamber 16. Additionally, a roller clamp assembly 24 is mounted on IV pole 12 and is engaged with tubing 18 extending from drip chamber 16. In a step 306, the practitioner will then connect the IV bag 14 to the patient by inserting a luer (not shown) provided on an end of the tubing 18 extending from the drip chamber 16.

As shown in FIG. 7 in a next step 308, system 10 is actuated and programming provided in a processor 26a automatically calculates the required drip rate based on the patient's prescription entered into the HMI 28 and the infusion is started. In step 310 drop counter 20 counts the drops of fluid falling under gravity into drip chamber 16 over a preset period of time to determine the drip rate of the fluid. Drop counter 20 feeds the drip rate data to the processor 26a. In step 312, the programming of processor 26a determines if the desired drip rate has been reached by comparing the drip rate measured by the drop counter 20 with the prescribed drip rate calculated in step 308. If the answer to the query of step 312 is “No” then two pathways are possible in the method as described below.

In a first pathway, in step 314, processor 26a automatically adjusts the position of the roller clamp 46 of roller clamp assembly 24 to reach the appropriate drip rate. The system 10 automatically checks the effect of the adjustment in the position of roller clamp 46 as indicated at 316. In this check, steps 310 and 312 are automatically repeated by system 10 until the desired drip rate is reached in step 312. In other words, the automatic adjustment of roller clamp and automatic checking of the effect thereof is repeated until the answer to the query, in step 312, is “Yes”. When this occurs, because the desired drip rate is reached, the infusion proceeds and the system monitors, in step 320, whether or not the total volume to be infused (VTBI) is reached. If the answer to that query is “No” then steps 310, 312, and 318 are automatically repeated by system 10 until the answer to the query in step 320 is “Yes”. If the VTBI is reached, in step 322 an alarm/alert will be issued to notify the practitioner that the infusion is completed. The alarm may be an audible sound and/or a visual message or image on HMI 28 and/or on computing system 30. Once the practitioner receives the alarm/alert they will disconnect the patient from tubing 18 and therefore from the IV bag.

A second pathway in response to the query in step 312 being “No” is indicated in step 318. If the desired drip rate has not been reached or has changed in some way this may be due to a potential problem in system 10 that cannot be solved by adjusting the position of the roller clamp in step 314. Such potential problems include but are not limited to issues relating to backflow, bubbles, or air in the line, and/or high pressure in the line. In reality, the system 10 periodically and/or continuously monitors for these potential issues during an infusion regardless of the answer to the query in step 312 being “Yes” or “No”. The fluid level in drip chamber 16 is periodically or continuously monitored by system 10. In particular, the camera 34 or a non-contact fluid level sensor registers the fluid level in drip chamber 16 and backflow will be detected by the system if the fluid level in drip chamber 16 rises above a preset limit. Camera 34 or other sensors may be utilized to monitor for bubbles or air in the line. Pressure sensors may be provided in system to monitor for changes in the pressure within the tubing 18. The periodic and/or continuous monitoring of the infusion is also represented by step 318 of the method 300. If no potential problems are detected during normal monitoring of the infusion or if the answer to the query in step 312 whether backflow, bubbles, air in the line, and/or high pressure are detected is “No”, then the method shown in FIG. 7 returns to step 310 and proceeds to step 312 and ultimately to steps 320 and 322 as described earlier herein.

If, however, backflow, bubbles or air in the line, or high pressure are detected during regular monitoring of system 10 or in response to the query in step 318 being “Yes”, then in step 324 an alert is issued. Step 326 shows that the practitioner is notified of the detected problem on their HMI 28 and the practitioner will then make decisions regarding the detected issue. It should be noted that visual feed of the system may be provided to the practitioner on the HMI 28 as part of the alert 324. If, for example, high pressure is detected in the line (i.e., tubing 18) in step 318, the practitioner may check the tubing 18 for a blockage and, if a blockage is found, will either clear the blockage or replace the tubing. Once the practitioner has made the necessary decisions and taken any necessary action in step 326, the method returns to step 31, then proceeds to step 312, and ultimately proceeds to steps 320 and 322 as described earlier herein.

As is evident from FIG. 7, the practitioner's only initial involvement in method 300 is entering the patient data, setting up the IV bag 14, drip chamber 16, tubing 18, drop counter 20, and roller clamp 46, and then starting the infusion process. Provided the entered patient data is accurate, system 10 will automatically deliver the prescribed fluid volume over the prescribed time period. If backflow or some other problem is detected during the infusion, then the system 10 will alert the practitioner to take the needed action to resolve the detected problem. Otherwise, the next involvement by the practitioner with system 10 is when the tubing 18 is removed from the patient. In view of the method 300 described above, it will be understood that the accuracy of infusion utilizing system 10 is greatly improved over the PRIOR ART method 200 and the possibility for human error in the infusion process is greatly decreased. Still further, the presently disclosed method 300 enables a single medical practitioner to oversee the infusion of multiple patients substantially at the same time.

In summary, a method of controlling a flow rate of a fluid to a patient “P” using an infusion system 10 including tubing 18 extending from an intravenous bag 14 includes entering patient data into a human-machine interface (HMI) 28 of the infusion system 10; receiving, at a processor 26a of the infusion system 10 the patient data from the HMI and drip rate data from a drop counter 20 of the infusion system 10; analyzing the patient data and the drip rate data with the programming of processor 26a; determining, with the programming of processor 26a, an appropriate dosage of fluid to be delivered to patient “P” and therefore a desired flow rate of fluid to be delivered by the infusion system 10 to the patient “P” over a period of time. The method further includes automatically adjusting a position of a roller clamp 46 of the infusion system 10 in real time using a drive mechanism 54 functionally linked to the processor 26a; and delivering the desired appropriate dosage of fluid to the patient “P” via the tubing 18 from intravenous bag 14. It should be noted that drop counter 20 continuously monitors the drip rate in drip chamber 16 in real time and continuously feeds the drip rate data to the processor 26a which then continuously analyzes the data in real time and automatically adjusts the position of the roller 46h of the roller clamp 46 in real time to provide the appropriate fluid dosage to the patient in real time. The HMI 28 may be functionally linked to a remote centralized computing system 30 configured to simultaneously monitor several identical systems, each of which is being used to infuse a different patient.

Automatically adjusting a position of a roller 46h of roller clamp 46 includes rotating the roller 46h in one of a first direction and a second direction by rotating pinion gear 54j in direction “A” or direction “B” and subsequently moving rack support 54a in the direction “C” or “D” accordingly. The automatic adjustment of roller 46h also includes contacting tubing 18 which extends through a bore 46c of the roller clamp 46 with the roller 46h; changing a size of the bore 18a of the tubing 18 as the roller 46h rotates in the one of the first direction and the second direction; and changing the flow rate of the fluid through the tubing 18 to the desired flow rate as the size of the bore 18a is changed.

Receiving drip rate data from the drop counter 20 includes counting a number of drops “F1”, “F2” of fluid entering the drip chamber 16 from an intravenous bag 14 over a preset period of time; communicating the counted number of drops “F1”, “F2” from the drop counter 20 to the processor on PCB 26; determining, with programming in the processor, a flow rate of fluid moving through the tubing 18 from the drip chamber 16; and automatically adjusting the position of the roller 46h relative to the roller clamp 46 without human intervention. If the flow rate of fluid from IV bag 14 into drip chamber 16 changes, the rate of change will be detected by drop counter 20 and will be sent on to the processor on PCB 26. The processor will analyze the patient data in light of the drip rate data, will recalculate the required flow rate through the tubing and thereby any required change in the roller 46h that is needed to attain or maintain the desired flow rate of fluid to the patient “P”. If it is determined via the programming that the roller 46h needs to be moved upwardly or downwardly relative to roller clamp 46, the drive mechanism 54 will be actuated, motor 56 will drive pinion gear 54j in the needed first direction “A” or second direction “B”; rotation of the pinion gear 54j will cause the rack support 54a of the drive mechanism 54 to move in the associated direction “C” or “D”, as needed in response to rotation “A” or “B”; and therefore roller 46h will rotate into the calculated position and affect the flow rate of fluid through tubing 18 accordingly. This adjustment in the position of roller 46h relative to roller clamp 46 will occur automatically and without human intervention.

As discussed above, previously known gravity infusion systems have required medical practitioners to personally determine the setting of a roller clamp to deliver a desired dosage of fluid to a patient. Human error could result in the patient being given too high a dosage or too low a dosage of fluid with the previously-known methodology of dosage control. System 10 disclosed herein automates gravity infusion and thereby helps to decrease the likelihood of human error in the delivery of fluid to a patient. System 10 also helps to increase the likelihood that a correct dosage of fluid will be delivered to a patient. Since multiple identical systems can be simultaneously monitored and controlled, efficiency and accuracy is increased, and a practitioner is able to more effectively care for multiple patients at the same time.

System 10 also enables more accurate infusion than was possible with previously-known gravity infusion systems. Drip rate from IV bags is constantly changing due to tube creep, movement of the patient, changes in fluid bag height etc. Therefore a practitioner would previously have had to continuously check or monitor the drip rate and make appropriate adjustments to the position of the roller clamp. System 10, by contrast, is a closed loop system which continuously monitors drip feed rate and automatically makes position adjustments of roller clamp 46 based on real-time drip rates. Because of this continuous monitoring and adjustment, system 10 is able to ensure accurate infusion rates.

As described herein, aspects of the present disclosure may include one or more electrical, pneumatic, hydraulic, or other similar secondary components and/or systems therein. The present disclosure is therefore contemplated and will be understood to include any necessary operational components thereof. For example, electrical components will be understood to include any suitable and necessary wiring, fuses, or the like for normal operation thereof. Similarly, any pneumatic systems provided may include any secondary or peripheral components such as air hoses, compressors, valves, meters, or the like. It will be further understood that any connections between various components not explicitly described herein may be made through any suitable means including mechanical fasteners, or more permanent attachment means, such as welding or the like. Alternatively, where feasible and/or desirable, various components of the present disclosure may be integrally formed as a single unit.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of technology disclosed herein may be implemented using hardware, software, or a combination thereof. When implemented in software, the software code or instructions can be executed on any suitable processor or collection of processors, whether provided in a single computer or distributed among multiple computers. Furthermore, the instructions or software code can be stored in at least one non-transitory computer readable storage medium.

Also, a computer or smartphone utilized to execute the software code or instructions via its processors may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers or smartphones may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software/instructions that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, USB flash drives, SD cards, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

The terms “program” or “software” or “instructions” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

All definitions, as defined and used herein, should be understood to control over dictionary definitions, definitions in documents incorporated by reference, and/or ordinary meanings of the defined terms.

“Logic”, as used herein, includes but is not limited to hardware, firmware, software, and/or combinations of each to perform a function(s) or an action(s), and/or to cause a function or action from another logic, method, and/or system. For example, based on a desired application or needs, logic may include a software controlled microprocessor, discrete logic like a processor (e.g., microprocessor), an application specific integrated circuit (ASIC), a programmed logic device, a memory device containing instructions, an electric device having a memory, or the like. Logic may include one or more gates, combinations of gates, or other circuit components. Logic may also be fully embodied as software. Where multiple logics are described, it may be possible to incorporate the multiple logics into one physical logic. Similarly, where a single logic is described, it may be possible to distribute that single logic between multiple physical logics.

Furthermore, the logic(s) presented herein for accomplishing various methods of this system may be directed towards improvements in existing computer-centric or internet-centric technology that may not have previous analog versions. The logic(s) may provide specific functionality directly related to structure that addresses and resolves some problems identified herein. The logic(s) may also provide significantly more advantages to solve these problems by providing an exemplary inventive concept as specific logic structure and concordant functionality of the method and system. Furthermore, the logic(s) may also provide specific computer implemented rules that improve on existing technological processes. The logic(s) provided herein extends beyond merely gathering data, analyzing the information, and displaying the results. Further, portions or all of the present disclosure may rely on underlying equations that are derived from the specific arrangement of the equipment or components as recited herein. Thus, portions of the present disclosure as it relates to the specific arrangement of the components are not directed to abstract ideas. Furthermore, the present disclosure and the appended claims present teachings that involve more than performance of well-understood, routine, and conventional activities previously known to the industry. In some of the method or process of the present disclosure, which may incorporate some aspects of natural phenomenon, the process or method steps are additional features that are new and useful.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some holsters and disjunctively present in other holsters. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, the term “effecting” or a phrase or claim element beginning with the term “effecting” should be understood to mean to cause something to happen or to bring something about. For example, effecting an event to occur may be caused by actions of a first party even though a second party actually performed the event or had the event occur to the second party. Stated otherwise, effecting refers to one party giving another party the tools, objects, or resources to cause an event to occur. Thus, in this example a claim element of “effecting an event to occur” would mean that a first party is giving a second party the tools or resources needed for the second party to perform the event, however the affirmative single action is the responsibility of the first party to provide the tools or resources to cause said event to occur.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” maybe used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present disclosure.

An embodiment is an implementation or example of the present disclosure. Reference in the specification to “an embodiment,” “one embodiment,” “some embodiments,” “one particular embodiment,” “an exemplary embodiment,” or “other embodiments,” or the like, means that a particular feature, structure, or characteristic described in connection with the embodiments is included in at least some embodiments, but not necessarily all embodiments, of the disclosure. The various appearances “an embodiment,” “one embodiment,” “some embodiments,” “one particular embodiment,” “an exemplary embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” maybe used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, the method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

To the extent that the present disclosure has utilized the term “invention” in various titles or sections of this specification, this term was included as required by the formatting requirements of word document submissions pursuant to guidelines/requirements of the United States patent and Trademark Office and shall not, in any manner, be considered a disavowal of any subject matter.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed:

1. An infusion system comprising:
a drop counter adapted to be engaged with a drip chamber of a fluid source, wherein the fluid source is configured to deliver a fluid to a patient's body through tubing under force of gravity;
a roller clamp functionally linked to the drop counter;
an adjustment assembly operably engaged with a roller of the roller clamp, said adjustment assembly including at least one spring-loaded plunger which urges the roller towards the tubing; and
a processor provided with programming configured to automatically adjust a position of the roller relative to the roller clamp via the adjustment assembly and at least partially in response to drip data gathered by the drop counter.

2. The infusion system according to claim 1, wherein the at least one spring-loaded plunger comprises a first plunger and a second plunger which engage spaced apart locations on the roller, and wherein the first plunger and the second plunger, together, push and rotate the roller relative to the roller clamp.

3. The infusion system according to claim 2, wherein the first plunger and the second plunger are independently operable.

4. The infusion system according to claim 2, wherein each of the first plunger and the second plunger includes a pin roller which contacts a circumferential surface of the roller and an exterior surface of the pin roller which contacts the circumferential surface of the roller includes a resilient material.

5. The infusion system according to claim 1, further comprising a housing with a holster provided therein, wherein the holster is configured to receive any one of a variety of differently configured roller clamps therein.

6. The infusion system according to claim 1, wherein the fluid source includes an intravenous bag adapted to contain a volume of the fluid and the drip chamber is operatively engaged with the intravenous bag; and wherein the drop counter is configured to automatically count drops of fluid falling into the drip chamber from the intravenous bag and feed drip data to the processor in real time.

7. The infusion system according to claim 1, further comprising a human-machine interface (HMI) functionally linked with the processor, said HMI being configured to enable access patient data.

8. The infusion system according to claim 1, further comprising a camera provided on the drop counter, said camera being configured to capture one or both of drip data and fluid level within a drip chamber of the drop counter.

9. An infusion system comprising:
an intravenous bag adapted to hold a volume of fluid to be delivered to a patient;
tubing extending between the intravenous bag and the patient's body;
a drip chamber engaged with the tubing at a location between the intravenous bag and the patient's body;
a drop counter operably engaged with the drip chamber, said drop counter being configured to automatically determine a number of drops of fluid entering the drip chamber from the intravenous bag in real time;
a roller clamp positioned between the drip chamber and the patient's body, wherein the tubing extends through a bore of the roller clamp;
a roller provided in the roller clamp, wherein the roller bears upon the tubing extending through the bore of the roller clamp;
a first plunger and a second plunger which contact the roller at spaced-apart locations from one another, said first plunger and the second plunger being operable to move the roller relative to the roller clamp; and
a processor functionally linked to drop counter and the roller, said processor automatically controlling movement of the roller relative to the tubing to control a flow rate of fluid through the tubing in response to drip data fed by the drop counter to the processor.

10. The infusion system according to claim 9, wherein the first plunger and the second plunger are independently operable.

11. The infusion system according to claim 9, further comprising:
a human-machine interface (HMI) functionally linked with the processor, said HMI being configured to access patient data and provide the same to the processor; and
a remote computing system functionally linked with the HMI.

12. The infusion system according to claim 9, further comprising a camera provided on the drop counter, wherein the camera is configured to capture images of the number of drops of fluid entering a drip chamber of the drop counter and or a fluid level within the drip chamber.

13. A method of controlling a flow rate of a fluid to a patient using an infusion system which includes tubing extending from an intravenous bag; said method comprising:
accessing patient data via a human-machine interface (HMI) of the infusion system;
receiving, at a processor of the infusion system, drip rate data from a drop counter of the infusion system;
analyzing the patient data and the drip rate data with the processor;
determining, with the processor, a desired flow rate of fluid based on a desired dosage of fluid to be delivered by the infusion system to the patient over a period of time based on the analysis of the patient data and drip rate data;
automatically adjusting a position of a roller of a roller clamp of the infusion system using a drive mechanism functionally linked to the processor to push and rotate the roller relative to the roller clamp; and
delivering the desired dosage of fluid to the patient through the tubing.

14. The method according to claim 13, wherein automatically adjusting a position of a roller clamp includes:
rotating a roller of the roller clamp in one of a first direction and a second direction;
contacting the tubing which extends through a bore of the roller clamp with the roller;
changing a size of the bore of the tubing as the roller rotates in the one of the first direction and the second direction; and
changing the flow rate of the fluid through the tubing to the desired flow rate as the size of the bore is changed.

15. The method according to claim 13, wherein receiving drip rate data from the drop counter includes:
automatically counting, with a sensor of the drop counter, a number of drops of fluid entering a drip chamber from an intravenous bag in real time;
communicating the counted number of drops of fluid from the drop counter to the processor;
determining, with programming in the processor, a flow rate of fluid moving through the tubing from the drip chamber; and
automatically adjusting the position of the roller relative to the clamp without human intervention and in real time.

16. The method according to claim 13, wherein receiving drip rate data from the drop counter includes:
capturing images of drops of fluid entering the drip chamber of the drop counter with a camera; and
determining the drip rate data from the captured images.

17. The method according to claim 13, further comprising:
capturing images of an actual level of fluid within a drip chamber of the drop counter; and
determining when the actual level of fluid rises above a threshold fluid level.

18. The method according to claim 13, further comprising:
determining backflow within a drip chamber of the drop counter when the actual level of fluid rises above the threshold fluid level; and
issuing an alarm.

19. The method according to claim 13, further comprising:
providing a sensor for detecting one of backflow, bubbles in the fluid, air in the fluid, and changes in pressure in the tubing; and
generating one or both of an audible alarm and a visual alarm when the sensor detects the one of backflow, bubbles in the fluid, air in the fluid, high pressure in the tubing and a low pressure in the tubing.

20. A system for controlling a flow rate of fluid to a patient through tubing extending from an intravenous bag; said system comprising:
a drop counter operably engaged with a drip chamber provided between the intravenous bag and the tubing;
a roller clamp in electronic communication with the drop counter; wherein the roller clamp includes:
a bore through which the tubing is received;
a roller extending into the bore and contacting the tubing;
a drive mechanism operable to push and rotate the roller in one of first direction and a second direction;
a processor in communication with the drive mechanism; and
a human-machine interface (HMI) functionally linked with the processor.
